(19) United States
(12) Patent Application Publication
(10) Pub. No.: US 2023/0346815 A1
(43) Pub. Date: Nov. 2, 2023

(54) LMP7-SELECTIVE INHIBITORS FOR THE TREATMENT OF BLOOD DISORDERS AND SOLID TUMORS

(71) Applicant: Merck Patent GmbH, Darmstadt (DE)

(72) Inventors: Christina ESDAR, Darmstadt (DE); Manja FRIESE-HAMIM, Darmstadt (DE); Michael SANDERSON, Darmstadt (DE); Gina WALTER-BAUSCH, Darmstadt (DE)

(73) Assignee: Merck Patent GmbH, Darmstadt (DE)

(21) Appl. No.: 18/247,739

(22) PCT Filed: Oct. 5, 2021
(86) PCT No.: PCT/EP2021/077427

§ 371 Date: Apr. 3, 2023

Related U.S. Application Data
(60) Provisional application No. 63/087,516, filed on Oct. 5, 2020.

(51) Int. Cl. A61K 31/69; A61P 35/00; A61K 45/06

(52) U.S. Cl. CPC A61K 31/69; A61P 35/00; A61K 45/06

(57) ABSTRACT

Alpha-amino boronic acid derivatives are useful for selectively inhibiting the activity of immunoproteasome subunit LMP7 and for the treatment of medical conditions affected by immunoproteasome activity such as blood disorders and solid tumors, which are defined by specific genetic alterations and/or inadequate responsiveness to other therapeutic treatments. In particular, the compounds disclosed herein are selective LMP7 inhibitors, which may be useful alone or in combination for the treatment of blood disorders, such as multiple myeloma, and certain solid tumors.

### FIELD OF THE INVENTION

The present invention relates to use of α-amino boronic acid derivatives which are useful for inhibiting the activity of immunoproteasome (LMP7) and for the treatment of medical conditions affected by immunoproteasome activity such as blood disorders and solid tumors. In particular, the compounds of the present invention are selective immunoproteasome inhibitors which may be useful alone, or in combination for the treatment of blood disorders, such as subjects with multiple myeloma who have a t(4;14) or t(14;16) translocation, and certain solid tumors which have genetic alterations and/or are insufficiently responding to other therapeutic treatments.

### BACKGROUND OF THE INVENTION

The proteasome (also known as macropain, the multicatalytic protease, and 20S protease) is a high molecular weight, multi-subunit protease which has been identified in every examined species from an archaebacterium to human. The enzyme has a native molecular weight of approximately 650,000 and, as revealed by electron microscopy, a distinctive cylinder-shaped morphology (Orlowski, 1990; Rivett, 1989). The proteasome subunits range in molecular weight from 20,000 to 35,000 and are homologous to one another, but not to any other known protease.

The 20S proteasome is a 700 kDa cylindrical-shaped multi-catalytic protease complex comprised of 28 subunits, classified as α- and β-type, that are arranged in 4 stacked heptameric rings. In yeast and other eukaryotes, 7 different α subunits form the outer rings and 7 different β subunits comprise the inner rings. The α subunits serve as binding sites for regulatory complexes such as 19S (PA700), as well as a physical barrier for the inner proteolytic chamber formed by the two β subunit rings. Thus, in cells, the proteasome is believed to exist as a 26S particle (“the 26S proteasome”).

Experiments have shown that inhibition of the 20S form of the proteasome can be readily correlated to inhibition of 26S proteasome.

Cleavage of amino-terminal prosequences of β subunits during particle formation expose amino-terminal threonine residues, which serve as the catalytic nucleophiles. The subunits responsible for catalytic activity in proteasome thus possess an amino terminal nucleophilic residue, and these subunits belong to the family of N-terminal nucleophile (Ntn) hydrolases (where the nucleophilic N-terminal residue is, for example, Cys, Ser, Thr, and other nucleophilic moieties). This family includes, for example, penicillin G acylase (PGA), penicillin V acylase (PVA), glutamine PRPP amidotransferase (GAT), and bacterial glycosylasparaginase. In addition to the ubiquitously expressed β subunits, higher vertebrates also possess three interferon-γ-inducible β subunits (LMP7, LMP2 and MECL-1), which replace their normal counterparts, β5, β1 and β2, respectively.

When all three IFN-γ-inducible subunits are present, the proteasome is referred to as an “immunoproteasome”. Thus, eukaryotic cells can possess two forms of proteasomes in varying ratios.

Through the use of different peptide substrates, three major proteolytic activities have been defined for the eukaryote 20S proteasomes: chymotrypsin-like activity (CT-L), which cleaves after large hydrophobic residues; trypsin-like activity (T-L), which cleaves after basic residues; and peptidylglutamyl peptide hydrolyzing activity (PGPH), which cleaves after acidic residues. Two additional less characterized activities have also been ascribed to the proteasome: BrAAP activity, which cleaves after branched-chain amino acids; and SNAAP activity, which cleaves after small neutral amino acids. Although both forms of the proteasome possess all five enzymatic activities, differences in the extent of the activities between the forms have been described based on specific substrates. For both forms of the proteasome, the major proteasome proteolytic activities appear to be contributed by different catalytic sites within the 20S core.

In eukaryotes, protein degradation is predominately mediated through the ubiquitin pathway in which proteins targeted for destruction are ligated to the 76 amino acid polypeptide ubiquitin. Once targeted, ubiquitinated proteins then serve as substrates for the 26S proteasome, which cleaves proteins into short peptides through the action of its three major proteolytic activities. While having a general function in intracellular protein turnover, proteasome-mediated degradation also plays a key role in many processes such as major histocompatibility complex (MHC) class I presentation, apoptosis and cell viability, antigen processing, NF-κB activation, and transduction of pro-inflammatory signals.

Proteasome activity is high in muscle wasting diseases that involve protein breakdown such as muscular dystrophy, cancer and AIDS. Proteasomes also generate peptides for presentation as antigens on class I MHC molecules, thus forming an essential component of the adaptive immune system (Goldberg and Rock, 1992).

Proteasomes are involved in neurodegenerative diseases and disorders such as amyotrophic lateral sclerosis (ALS) (Allen et al., 2003; Puttaparthi and Elliott, 2005), Sjogren Syndrome (Egerer et al., 2006), systemic lupus erythematoses and lupus nephritis (SLE/LN) (Ichikawa et al., 2012; Lang et al., 2010; Neubert et al., 2008), glomerulonephritis (Bontscho et al., 2011), rheumatoid arthritis (van der Heijden et al., 2009), inflammatory bowel disease (IBD), ulcerative colitis, Crohn's diseases (Basler et al., 2010; Inoue et al., 2009; Schmidt et al., 2010), multiple sclerosis (Elliott et al., 2003; Fissolo et al., 2008; Hosseini et al., 2001; Vanderlugt et al., 2000), amyotrophic lateral sclerosis (ALS) (Allen et al., 2003; Puttaparthi and Elliott, 2005), osteoarthritis (Ahmed et al., 2012; Etienne et al., 2008), atherosclerosis (Feng et al., 2010), psoriasis (Kramer et al., 2007), myasthenia gravis (Gomez et al., 2011), dermal fibrosis (Fineschi et al., 2006; Koca et al., 2012; Mutlu et al., 2012), renal fibrosis (Sakairi et al., 2011), cardiac fibrosis (Ma et al., 2011), liver fibrosis (Anan et al., 2006), lung fibrosis (Fineschi et al., 2006), imunoglobulin A nephropathy (IgA nephropathy) (Coppo et al., 2009), vasculitis (Bontscho et al., 2011), transplant rejection (Waiser et al., 2012), hematological malignancies (Chen et al., 2011; Singh et al., 2011) and asthma (Nair et al., 2017).

However, it should be noted that approved proteasome inhibitors including bortezomib, carfilzomib and ixazomib inhibit both the constitutive proteasome and immunoproteasome and thus are considered “pan-proteasome inhibitors” (Altun et al., 2005). Furthermore, pan-proteasome inhibitors have been described to inhibit non-proteasome associated proteases, which may contribute to their adverse toxicity profiles (Arastu-Kapur et al., 2011).

In addition to conventional pan-proteasome inhibitors, a novel approach may be to specifically target the hematological-specific immunoproteasome, thereby increasing overall effectiveness and reducing negative off-target effects. It has been shown that immunoproteasome is highly expressed in multiple myeloma; a malignancy of plasma cells. Despite the emergence of new therapeutic modalities such as pan-proteasome inhibitors (e.g. bortezomib, carfilzomib, ixazomib), many multiple myeloma patients are refractory to treatment or develop resistance (Manier et al., 2017; Pawlyn and Morgan, 2017; Sonneveld et al., 2016). In particular, multiple myeloma patients harboring the ‘high-risk’ cytogenetic abnormalities/translocations t(4;14) or t(14;16) demonstrate especially poor prognosis (Manier et al., 2017; Pawlyn and Morgan, 2017; Sonneveld et al., 2016). The high-risk t(4;14) cytogenetic abnormality/translocation results in deregulated expression of the genes fibroblast growth factor receptor 3 (FGFR3) and multiple myeloma SET domain (MMSET) (Manier et al., 2017; Sonneveld et al., 2016). Positivity for t(4;14) is detected in approximately 15% of multiple myeloma patients and is associated with adverse/poor prognosis (Manier et al., 2017; Pawlyn and Morgan, 2017; Sonneveld et al., 2016). Furthermore, positivity for t(4;14) confers a high-risk of patients progressing from the premalignant states of monoclonal gammopathy of undetermined significance (MGUS) and smouldering myeloma (SMM) to multiple myeloma, which is malignant (Bustoros et al., 2017). Many multiple myeloma patients harboring the t(4;14) translocation are refractory to treatment with therapies such as pan-proteasome inhibitors, or they develop resistance and undergo disease relapse (Manier et al., 2017; Pawlyn and Morgan, 2017).

The high-risk t(14;16) cytogenetic abnormality/translocation is present in approximately 5% of multiple myeloma patients and leads to deregulated expression of the MAF bZIP transcription factor (MAF) (Manier et al., 2017; Pawlyn and Morgan, 2017). Multiple myeloma patients positive for t(14;16) demonstrate adverse/poor prognosis (Manier et al., 2017; Pawlyn and Morgan, 2017; Sonneveld et al., 2016). Many multiple myeloma patients harboring the t(14;16) translocation are refractory to treatment with therapies such as pan-proteasome inhibitors, or they develop resistance and undergo disease relapse (Manier et al., 2017; Pawlyn and Morgan, 2017). In particular, deregulated expression of MAF, or the related gene MAFB, has been described to confer resistance of multiple myeloma cells to pan-proteasome inhibitors (Qiang et al., 2016; Qiang et al., 2018).

Furthermore, resistance or refractoriness in multiple myeloma to drugs such as pan-proteasome inhibitors (e.g. bortezomib, carfilzomib, ixazomib) has been described to be mediated by gene mutation, deregulated gene expression and/or gene dependency (herein described as “genetic alteration(s)”) for specific genes or pathways such as IRF4, XPO1, MAX, MAF, MAFB, MCL1, FGFR3, IGF1R, CDKN2A, EGFR, Wnt/β-Catenin pathway (e.g. APC, WNT1, WNT5B), NFκB pathway (e.g. NFKB1), ubiquitination pathway (e.g. UBA52, MEDS), MAPK pathway (e.g. KRAS, NRAS, HRAS, BRAF, MAP4K3, NF1) and/or DNA repair pathway (e.g. TP53, ATM) (Bustoros et al., 2017; Chanukuppa et al., 2019; Chong et al., 2015; Jin et al., 2019; Kortum et al., 2016; Park et al., 2014; Podar et al., 2008; Savvidou et al., 2017; Tron et al., 2018; Turner et al., 2016; Yang et al., 2018; Zhang et al., 2016; https://depmap.org/portal/).

Finally, the treatment of multiple myeloma patients with pan-proteasome inhibitors (e.g. bortezomib, carfilzomib, ixazomib) has been shown to lead to an incomplete duration of suppression of the proteasomal subunits including large multifunctional peptidase 7 (LMP7, β5i, PSMB8) (Assouline et al., 2014; Lee et al., 2016), which may potentially limit the therapeutic effectiveness of these drugs in multiple myeloma patients.

Despite the use of therapeutic modalities such as pan-proteasome inhibitors (e.g. bortezomib, carfilzomib, ixazomib), many multiple myeloma patients, in particular those harboring the high-risk cytogenetic abnormalities/translocations t(4;14) and/or t(14;16), and who carry specific genetic alterations, may be refractory to, or develop resistance to, current therapy. Furthermore, the incomplete duration of inhibition of LMP7 described with pan-proteasome inhibitors may potentially be associated with reduced therapeutic effectiveness of these agents in multiple myeloma patients.

Therefore, differentiated therapeutic agents demonstrating one or more of the following advantages are critically needed to improve the prognosis of multiple myeloma patients:
1) activity in subjects with a blood disorder and/or solid tumors exhibiting resistance and/or refractoriness to therapies such as pan-proteasome inhibitors (e.g. bortezomib, carfilzomib, ixazomib);
2) activity superior to therapies such as pan-proteasome inhibitors in subjects with a blood disorder, including multiple myeloma, who are positive for the translocation t(4;14) or t(14;16);
3) activity in subjects with a blood disorder (e.g., multiple myeloma) and/or solid tumors who are positive for specific genetic alterations that trigger refractoriness/resistance to therapies such as pan-proteasome inhibitors; and/or
4) more complete suppression of LMP7 and/or more complete modulation of other pharmacodynamic biomarkers (e.g., markers of tumor cell apoptosis) relevant to treatment of blood disorders (including multiple myeloma) and/or solid tumors compared to therapies such as currently available pan-proteasome inhibitors.

Furthermore, there is a pressing unmet need for treatments of subjects with cancer such as multiple myeloma or solid tumors with genetic alterations which make them less susceptible to treatment with standard of care therapeutic options.

### SUMMARY OF INVENTION

We have discovered an immunoproteasome-specific inhibitor, e.g., compound 9, or other compounds as described herein, which displays enhanced efficiency on cells from a hematologic origin which is useful for the treatment of blood disorders, such as multiple myeloma, and/or solid tumors, and possess one or more advantageous attributes listed above.

One embodiment of the invention is a method of treating a blood disorder comprising administering an effective amount of an LMP7-selective inhibitor of the invention to a subject in need thereof, wherein the subject has a t(4;14) or t(14;16) translocation.

Another embodiment of the invention is a method of treating cancer in a subject in need thereof, comprising administering an effective amount of an LMP7-selective inhibitor to the subject, wherein the subject has cancer with a genetic alteration.

In one aspect of either of the above two embodiments, the LMP7-selective inhibitor is selected from the list of compounds in Table 1, below. In another aspect of either of these two embodiments, the LMP7-selective inhibitor is compound 9:

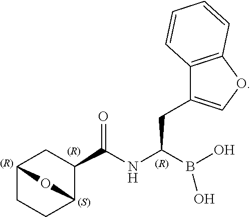

### BRIEF DESCRIPTION OF FIGURES

FIG. 1 shows increased in vivo anti-tumor activity of compound 9 compared to the pan-proteasome inhibitors bortezomib, carfilzomib and ixazomib in the t(4;14)-positive multiple myeloma model OPM-2.

FIG. 2 shows increased in vivo anti-tumor activity of compound 9 compared to the pan-proteasome inhibitors bortezomib and carfilzomib in the t(4;14)-positive multiple myeloma model NCI-H929.

FIG. 3 shows comparable activity of compound 9 as compared to the pan-proteasome inhibitor ixazomib in the t(4;14)-positive multiple myeloma model NCI-H929.

FIG. 4 shows the increased in vivo anti-tumor activity of compound 9 compared to the pan-proteasome inhibitors bortezomib and carfilzomib in the t(14;16)-positive multiple myeloma model MM.1S. The activity of compound 9 is comparable when compared to the pan-proteasome inhibitor ixazomib.

FIG. 5 shows the increased in vivo anti-tumor activity of compound 9 compared to the pan-proteasome inhibitors ixazomib and carfilzomib in the t(14;16)-positive multiple myeloma model RPMI 8826. The activity of compound 9 is comparable when compared to the pan-proteasome inhibitor bortezomib.

FIG. 6 shows in vivo treatment with compound 9 led to a significant reduction of enzymatic function of LMP7 in MM.1S multiple myeloma tumor cells compared to the control group, as indicated by the measurement of cleavage of the peptide (Ac-ANW)2R110. The effect of compound 9 on LMP7 activity was more pronounced and longer than that observed with the pan-proteasome inhibitors bortezomib, carfilzomib and ixazomib.

FIG. 7 shows in vivo treatment with compound 9 led to a significant induction of apoptosis in MM.1S multiple myeloma tumor cells compared to the control group, as indicated by the measurement of Caspase-3/-7 activity. The induction of apoptosis by compound 9 treatment was longer compared to that observed with the pan-proteasome inhibitors bortezomib, carfilzomib and ixazomib.

### DETAILED DESCRIPTION

The compounds described herein, as first reported in WO19/38250, are selective and potent inhibitors of the LMP7 proteolytic subunit of the immunoproteasome. This LMP7 selectivity distinguishes these compounds from pan-proteasome inhibitors (e.g. bortezomib, carfilzomib, ixazomib), which inhibit LMP7 and also other proteolytic subunits of both the immunoproteasome and constitutive proteasome.

Treatment of Blood Disorders

It has been surprisingly found that the highly potent and selective LMP7 inhibitor compound 9 demonstrated anti-tumor efficacy in several preclinical in vivo models of multiple myeloma that were refractory/resistant to the pan-proteasome inhibitors bortezomib, carfilzomib and/or ixazomib. This suggests that compound 9, and the LMP7-selective inhibitors described herein, could deliver a therapeutic benefit to patients with blood disorders such as multiple myeloma and/or solid tumors that are refractory, resistant, or exhibit a sub-optimal response to treatments such as pan-proteasome inhibitors.

One embodiment of the invention is a method of treating a blood disorder comprising administering an effective amount of an LMP7-selective inhibitor of the invention to a subject in need thereof, wherein the subject has a t(4;14) or t(14;16 translocation. In one aspect of this embodiment, the LMP7-selective inhibitor is selected from the list of compounds in Table 1, below. In another aspect of either of these embodiments, the LMP7-selective inhibitor is compound 9:

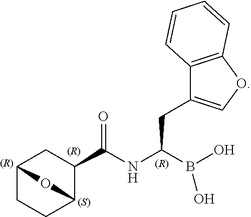

In one aspect of this embodiment, the blood disorder is a premalignant condition. In a further aspect of this embodiment, the premalignant blood disorder is monoclonal gammopathy of uncertain significance (MGUS); smoldering multiple myeloma (SMM); plasma cell leukemia; or solitary plasmacytoma.

In another aspect of this embodiment, the blood disorder is plasmacytoma and/or amyloid light-chain (AL) amyloidosis.

In another aspect of this embodiment, the blood disorder is a malignant condition. In a further aspect of this embodiment, the malignant blood disorder is multiple myeloma.

In any of the above embodiments and aspects of embodiments, the blood disorder may have a further genetic alteration. In one aspect of this embodiment, the genetic alteration is a gene mutation, dysregulated gene expression, and/or gene dependency. In one aspect of this embodiment, the genetic alteration is in specific genes or pathways such as IRF4, XPO1, MAX, MAF, MAFB, MCL1, FGFR3, IGF1R, CDKN2A, EGFR, Wnt/β-Catenin pathway (e.g. APC, WNT1, WNT5B), NFκB pathway (e.g. NFKB1), ubiquitination pathway (e.g. UBA52, MEDS), MAPK pathway (e.g. KRAS, NRAS, HRAS, BRAF, MAP4K3, NF1) and/or DNA repair pathway (e.g. TP53, ATM, BRCA1/2). In a further aspect of this embodiment, the genetic alteration is in one or more of the genes selected from APC, ARHGAP45, ASH2L, ATM, ATXN7, BRCA2, CCND2, CDC20, CDKN2A, CITED2, COQ6, DLST, DNAJC9, EGFR, EPC2, FGFR3, IGF1R, IRF2, IRF4, IRS1, KRAS, LYZ, MAF, MAP4K3, MAX, MCL1, MEDS, MEF2C, MMSET, MTA2, NFKB1, NRAS, NSD2, PIM2, POU2AF1, PSMC1, RAD21, RICTOR, RORA, SEC13, THY1, TP53, UBA52, WNT1, WNT5B, XPO1 and ZBTB38.

In an additional aspect of any of the above embodiments, the subject in need of treatment shows an incomplete and/or suboptimal response to the administration of one or more pan-proteasome inhibitor. In a further aspect of any of the above embodiments, the subject in need of treatment is resistant to treatment with one or more pan-proteasome inhibitors. In another aspect of any of the above embodiments, the subject in need of treatment is refractory to treatment with one or more pan-proteasome inhibitors. In any of the above aspects of the embodiments, the one or more pan-proteasome inhibitors is selected from the group consisting of bortezomib, carfilzomib, and ixazomib.

In another embodiment of the invention, the method of treating a blood disorder in a subject in need thereof comprises administering an LMP7-selective inhibitor of the invention in combination with of one or more additional therapeutic agents to the subject, wherein the subject has a t(4;14) or t(14;16) translocation. In one aspect of this embodiment, the one or more additional therapeutic agents is an EGFR pathway inhibitor, MAPK pathway inhibitor, XPO1 inhibitor, a DNA repair pathway inhibitor, FGFR pathway inhibitor, PI3K/AKT/mTOR pathway inhibitor, and/or MCL1 inhibitor.

In one aspect of the embodiment, the one or more additional therapeutic agents can include one or more therapeutic agents with the same and/or similar pathways. For illustration, if an LMP7-selective inhibitor of the invention is combined with an EGFR pathway inhibitor, the combination therapy could be an administration of compound 9 with pertuzumab and/or gefitinib. Likewise, a combination of an LMP7-selective inhibitor of the present invention can be combined with one or more additional therapeutic agents from multiple classes. For illustration, the combination may be administration of compound 9 with an EGFR pathway inhibitor, such as gefitinib, and a DNA repair pathway inhibitor, such as M3541. All possible permutations for combinations of the agents described herein represent specific aspects of the present invention.

In one aspect of the above embodiment, the EGFR pathway inhibitor is selected from erlotinib, afatinib, gefitinib, cetuximab, panitumumab, lapatinib, osimertinib, trastuzumab, and/or pertuzumab.

In another aspect of the above embodiment, the MAPK pathway inhibitor is selected from trametinib, cobimetinib, binimetinib, selumetinib, refametinib, pimasertib, AMG 510, MRTX849, vemurafenib, dabrafenib, encorafenib, LXH254, HM95573, XL281, RAF265, RAF709, LY3009120, ulixertinib, SCH772984, TNO155, RMC-4630, JAB-3068, JAB-3312, AMG-510, MRTX849, LY3499446 and/or BI 1701963.

In a further aspect of the above embodiment, the XPO1 inhibitor is selected from selinexor and/or KPT-8602.

In another aspect of the above embodiment, the DNA repair pathway inhibitor is selected from talazoparib, niraparib, olaparib, veliparib, rucaparib, pamiparib, AZD7648, M3814, CC-115, BAY1895344, AZD6738, M6620, M4344, M1774, M4076, M3541, AZD0157, AZD1390, prexasertib, GDC-0425, SRA-737, AZD1775 and/or Debio 0123.

In one aspect of the embodiment, the FGFR pathway inhibitor is selected from erdafitinib, AZD4547, LY2874455, Debio 1347, NVP-BGJ398, pemigatinib, rogaratinib, PRN1371, TAS-120, and/or nintedanib.

In a further aspect of the embodiment, the PI3K/AKT/mTOR pathway inhibitor is selected from rapamycin, temsirolimus, everolimus, ridaforolimus, alpelisib, idelalisib, copanlisib, duvelisib, MK-2206, and/or AZD5363.

In another aspect of the embodiment, the MCL1 inhibitor is selected from A-1210477, VU661013, AZD5991, AMG-176, AMG-397, S63845, S64315, venetoclax, HDM201, NVP-CGM097, RG-7112, MK-8242, RG-7388, SAR405838, AMG-232, DS-3032, RG7775, and/or APG-115.

In one aspect of any of the above embodiments, the LMP7-selective inhibitor is administered orally. Another aspect of any one of the above embodiments, the LMP7-selective inhibitor is administered once or twice daily. In one aspect of the above embodiments, the LMP7-selective inhibitor is administered once or twice per week. The invention encompasses both daily administration and intermittent administration (e.g., once or twice a week) on a regular schedule.

Treatment of Cancer

Another embodiment of the invention is a method of treating cancer comprising administering an effective amount of an LMP7-selective inhibitor of the invention to a subject in need thereof, wherein the cancer has one or more genetic alterations. In one aspect of this embodiment, the cancer is a solid tumor. In another aspect of this embodiment, the LMP7-selective inhibitor is selected from the compounds listed in Table 1. In another aspect of the embodiment, the LMP7-selective inhibitor is a compound according to formula (I):

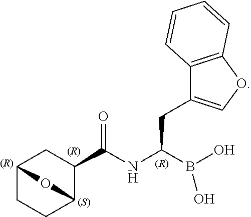

In one aspect of this embodiment, the cancer is linked to chronic inflammation.

In another aspect of this embodiment, the cancer with one or more genetic alterations is melanoma, glioma, glioblastomas, or cancer of the breast, lung, bladder, esophagus, stomach, colon, head, neck, ovary, prostate, pancreas, rectum, endometrium, or liver. In a further aspect of this embodiment, the cancer is selected from triple-negative breast cancer, non-small cell lung cancer, and head and neck carcinoma.

In another aspect of this embodiment, the cancer with one or more genetic alterations is a hematological malignancy. In a further aspect of this embodiment, the hematological malignancy is selected from mantle cell lymphoma (MCL), T cell leukemia/lymphoma, acute myeloid leukemia (AML), acute lymphoblastic leukemia (ALL), diffuse large B-cell lymphoma (DLBCL), chronic lymphocytic leukemia (CLL), chronic myelogenous leukemia (CML), follicular lymphoma (FL) or marginal zone B-cell lymphoma (MZL). In another aspect of this embodiment, the hematological malignancy is lymphoplasmacytic lymphoma, amyloid light chain amyloidosis (AL) and/or Walderstrom's macroglobulinemia (WM).

All aspects of these above embodiment include the treatment of subjects which have cancer with a genetic alteration. In one aspect of this embodiment, the genetic alteration is a gene mutation, dysregulated gene expression, and/or gene dependency. In one aspect of this embodiment, the genetic alteration is in specific genes or pathways such as IRF4, XPO1, MAX, MAF, MAFB, MCL1, FGFR3, IGF1R, CDKN2A, EGFR, Wnt/β-Catenin pathway (e.g. APC, WNT1, WNT5B), NFκB pathway (e.g. NFKB1), ubiquitination pathway (e.g. UBA52, MEDS), MAPK pathway (e.g. KRAS, NRAS, HRAS, BRAF, MAP4K3, NF1) and/or DNA repair pathway (e.g. TP53, ATM, BRCA1/2). In a further aspect of this embodiment, the genetic alteration is in one or more of the genes selected from APC, ARHGAP45, ASH2L, ATM, ATXN7, BRCA2, CCND2, CDC20, CDKN2A, CITED2, COQ6, DLST, DNAJC9, EGFR, EPC2, FGFR3, IGF1R, IRF2, IRF4, IRS1, KRAS, LYZ, MAF, MAP4K3, MAX, MCL1, MEDS, MEF2C, MMSET, MTA2, NFKB1, NRAS, NSD2, PIM2, POU2AF1, PSMC1, RAD21, RICTOR, RORA, SEC13, THY1, TP53, UBA52, WNT1, WNT5B, XPO1 and ZBTB38.

In an additional aspect of this embodiment, the subject in need of treatment shows an incomplete and/or suboptimal response to the administration of one or more pan-proteasome inhibitor. In a further aspect of this embodiment, the subject in need of treatment is resistant to treatment with one or more pan-proteasome inhibitors. In another aspect of this embodiment, the subject in need of treatment is refractory to treatment with one or more pan-proteasome inhibitors. In any of the above aspects of the embodiment, the one or more pan-proteasome inhibitors is selected from the group consisting of bortezomib, carfilzomib, and ixazomib.

In another embodiment of the invention, the method of treating a blood disorder in a subject in need thereof comprises administering an LMP7-selective inhibitor of the invention in combination with of one or more additional therapeutic agents to the subject. In one aspect of this embodiment, the one or more additional therapeutic agents is an EGFR pathway inhibitor, MAPK pathway inhibitor, XPO1 inhibitor, a DNA repair pathway inhibitor, FGFR pathway inhibitor, PI3K/AKT/mTOR pathway inhibitor, and/or MCL1 inhibitor.

In one aspect of the embodiment, the one or more additional therapeutic agents can include one or more therapeutic agents with the same and/or similar pathways. For illustration, if an LMP7-selective inhibitor of the invention is combined with an EGFR pathway inhibitor, the combination therapy could be an administration of compound 9 with pertuzumab and/or gefitinib. Likewise, a combination of an LMP7-selective inhibitor of the present invention can be combined with one or more additional therapeutic agents from multiple classes. For illustration, the combination may be administration of compound 9 with an EGFR pathway inhibitor, such as gefitinib, and a DNA repair pathway inhibitor, such as M3541. All possible permutations for combinations of the agents described herein represent specific aspects of the present invention.

In one aspect of the above embodiment, the EGFR pathway inhibitor is selected from erlotinib, afatinib, gefitinib, cetuximab, panitumumab, lapatinib, osimertinib, trastuzumab, and/or pertuzumab.

In another aspect of the above embodiment, the MAPK pathway inhibitor is selected from trametinib, cobimetinib, binimetinib, selumetinib, refametinib, pimasertib, AMG 510, MRTX849, vemurafenib, dabrafenib, encorafenib, LXH254, HM95573, XL281, RAF265, RAF709, LY3009120, ulixertinib, SCH772984, TNO155, RMC-4630, JAB-3068, JAB-3312, AMG-510, MRTX849, LY3499446 and/or BI 1701963.

In a further aspect of the above embodiment, the XPO1 inhibitor is selected from selinexor and/or KPT-8602.

In another aspect of the above embodiment, the DNA repair pathway inhibitor is selected from talazoparib, niraparib, olaparib, veliparib, rucaparib, pamiparib, AZD7648, M3814, CC-115, BAY1895344, AZD6738, M6620, M4344, M1774, M4076, M3541, AZD0157, AZD1390, prexasertib, GDC-0425, SRA-737, AZD1775 and/or Debio 0123.

In one aspect of the embodiment, the FGFR pathway inhibitor is selected from erdafitinib, AZD4547, LY2874455, Debio 1347, NVP-BGJ398, pemigatinib, rogaratinib, PRN1371, TAS-120, and/or nintedanib.

In a further aspect of the embodiment, the PI3K/AKT/mTOR pathway inhibitor is selected from rapamycin, temsirolimus, everolimus, ridaforolimus, alpelisib, idelalisib, copanlisib, duvelisib, MK-2206, and/or AZD5363.

In another aspect of the embodiment, the MCL1 inhibitor is selected from A-1210477, VU661013, AZD5991, AMG-176, AMG-397, S63845, S64315, venetoclax, HDM201, NVP-CGM097, RG-7112, MK-8242, RG-7388, SAR405838, AMG-232, DS-3032, RG7775, and/or APG-115.

One aspect any of the above embodiments, the LMP7-selective inhibitor is administered orally. Another aspect of any one of the above embodiments, the LMP7-selective inhibitor is administered once or twice daily. In another aspect of the above embodiments, the LMP7-selective inhibitor is administered once or twice per week.

LMP7-Selective Inhibitors of the Invention

Unspecific inhibitors of the proteasome and the immunoproteasome (i.e. pan-proteasome inhibitors) like bortezomib, carfilzomib and ixazomib have demonstrated their clinical value in the indication of multiple myeloma. However, their non-selective mechanism is associated with diverse and pronounced adverse events (e.g. thrombocytopenia, neutropenia, peripheral neuropathy, cardiotoxicity) which limit clinical utility of these agents and commonly lead to dose-reductions or dose cessation and does not enable prolonged inhibition of targets such as LMP7.

The approach to come up with more selective inhibitors of the immunoproteasome (in particular the LMP7/β5i immunoproteasome subunit), in order to reduce major side effects has been previously described for PR-924, a 100-fold selective LMP7 inhibitor (Singh et al., 2011). The authors demonstrated the presence of high expression levels of the immunoproteasome in multiple myeloma. In support of this concept, the authors also described the effect of a selective inhibitor of the LMP7 subunit on the induction of cell death in multiple myeloma cell lines as well as CD138+ multiple myeloma primary patient cells without decreasing the viability of normal peripheral blood mononuclear cells (PBMCs) from healthy volunteers. Furthermore, PR-924 has demonstrated efficacy in preclinical models of bortezomib-refractory multiple myeloma, as well as models of other hematological malignancies (Niewerth et al., 2014). These published data support the application of selective LMP7 inhibitors in hematological malignancies beyond multiple myeloma and also in settings of pan-proteasome inhibitor-refractory multiple myeloma.

The LMP7-selective inhibitors of the invention are specific proteasome inhibitors, and thus may avoid one or more of the toxicities seen with pan-proteasome inhibitors, as described above.

The preclinical models described herein, which show improved response to compound 9 compared to pan-proteasome inhibitors, are positive for the high-risk t(4;14) or t(14;16) cytogenetic abnormalities/translocations. This suggests that compound 9, or other LMP7-selective inhibitors described herein, could deliver a therapeutic benefit to multiple myeloma patients that are positive for these high-risk t(4;14) or t(14;16) cytogenetic abnormalities/translocations. Furthermore, these discoveries suggest that multiple myeloma patients exhibiting positivity for the t(4;14) or t(14;16) cytogenetic abnormalities/translocations could derive a therapeutic benefit from the combination of an LMP7-selective inhibitor as described herein with drugs that target genes, proteins or pathways (e.g. MAF, MMSET, FGFR3) which are altered or become essential to multiple myeloma cells as a result of the t(4;14) or t(14;16) translocations.

The aforementioned preclinical models, which displayed improved response to compound 9 as compared to pan-proteasome inhibitors, exhibit genetic alterations in APC, ARHGAP45, ASH2L, ATM, ATXN7, BRCA2, CCND2, CDC20, CDKN2A, CITED2, COQ6, DLST, DNAJC9, EGFR, EPC2, FGFR3, IGF1R, IRF2, IRF4, IRS1, KRAS, LYZ, MAF, MAP4K3, MAX, MCL1, MEDS, MEF2C, MMSET, MTA2, NFKB1, NRAS, NSD2, PIM2, POU2AF1, PSMC1, RAD21, RICTOR, RORA, SEC13, THY1, TP53, UBA52, WNT1, WNT5B, XPO1 and/or ZBTB38. This suggests that compound 9, or other LMP7-selective inhibitors described herein, could deliver a therapeutic benefit to multiple myeloma patients that exhibit these alterations. Furthermore, these patients could derive a therapeutic benefit from the combination of an LMP7-selective inhibitor with drugs that target factors implicated in these genetic alterations (e.g., EGFR pathway inhibitors in patients harboring EGFR genetic alterations).

One embodiment of the invention is the use of compound 9, or another LMP7-selective inhibitor described herein, to treat subjects with multiple myeloma, MGUS, SMM or other malignancies that are resistant or refractory to standard therapies such as pan-proteasome inhibitors (e.g. bortezomib, carfilzomib, ixazomib). In another embodiment of the invention is the use of compound 9, or another LMP7-selective inhibitor described herein, to treat subjects with plasma cell leukemia, solitary plasmacytoma or amyloid light-chain (AL) amyloidosis. In another embodiment of the invention is the use of compound 9, or another LMP7-selective inhibitor described herein, to treat subjects with solid tumors.

Another embodiment of the invention is the use of compound 9, or another LMP7-selective inhibitor described herein, for treatment of subjects with multiple myeloma, MGUS, SMM or other malignancies that harbor the high-risk cytogenetic alterations/translocations t(4;14) and t(14;16). Assessment of these cytogenetic alterations/translocations can be performed by karyotyping, fluorescence in situ hybridization (FISH), nucleotide sequencing and/or other standard methodologies.

Another embodiment of the invention is the use of compound 9, or another LMP7-selective inhibitor described herein, for treatment of subjects with plasma cell leukemia, solitary plasmacytoma or amyloid light-chain (AL) amyloidosis that harbor the high-risk cytogenetic alterations/translocations t(4;14) and t(14;16).

One additional embodiment of the present invention is the use of compound 9, or another LMP7-selective inhibitor described herein, to treat subjects with multiple myeloma, MGUS, SMM or other malignancies that demonstrate genetic alteration in either of the following genes or pathways: APC, ARHGAP45, ASH2L, ATM, ATXN7, BRCA2, CCND2, CDC20, CDKN2A, CITED2, COQ6, DLST, DNAJC9, EGFR, EPC2, FGFR3, IGF1R, IRF2, IRF4, IRS1, KRAS, LYZ, MAF, MAP4K3, MAX, MCL1, MEDS, MEF2C, MMSET, MTA2, NFKB1, NRAS, NSD2, PIM2, POU2AF1, PSMC1, RAD21, RICTOR, RORA, SEC13, THY1, TP53, UBA52, WNT1, WNT5B, XPO1, ZBTB38, Wnt/β-Catenin pathway, NFκB pathway, DNA repair pathway, MAPK pathway and/or ubiquitination pathway. Genetic alteration is defined as genetic mutation, deregulated expression or dependency. The assessment of these genetic alterations can be performed by nucleotide sequencing, karyotyping, FISH, protein and/or RNA expression analyses, flow cytometry and/or other standard methodologies.

Another embodiment of the present invention is the use of compound 9, or another LMP7-selective inhibitor described herein, to treat subjects with plasma cell leukemia, solitary plasmacytoma, lymphoplasmacytic lymphoma, amyloid light-chain (AL) amyloidosis or Waldenström's macroglobulinemia (WM) that demonstrate genetic alteration in either of the following genes or pathways: APC, ARHGAP45, ASH2L, ATM, ATXN7, BRCA2, CCND2, CDC20, CDKN2A, CITED2, COQ6, DLST, DNAJC9, EGFR, EPC2, FGFR3, IGF1R, IRF2, IRF4, IRS1, KRAS, LYZ, MAF, MAP4K3, MAX, MCL1, MEDS, MEF2C, MMSET, MTA2, NFKB1, NRAS, NSD2, PIM2, POU2AF1, PSMC1, RAD21, RICTOR, RORA, SEC13, THY1, TP53, UBA52, WNT1, WNT5B, XPO1, ZBTB38, Wnt/β-Catenin pathway, NFκB pathway, DNA repair pathway, MAPK pathway and/or ubiquitination pathway.

A further embodiment of the present invention is the use of compound 9, or another LMP7-selective inhibitor described herein, to treat subjects with solid tumors that demonstrate genetic alteration in either of the following genes or pathways: APC, ARHGAP45, ASH2L, ATM, ATXN7, BRCA2, CCND2, CDC20, CDKN2A, CITED2, COQ6, DLST, DNAJC9, EGFR, EPC2, FGFR3, IGF1R, IRF2, IRF4, IRS1, KRAS, LYZ, MAF, MAP4K3, MAX, MCL1, MEDS, MEF2C, MMSET, MTA2, NFKB1, NRAS, NSD2, PIM2, POU2AF1, PSMC1, RAD21, RICTOR, RORA, SEC13, THY1, TP53, UBA52, WNT1, WNT5B, XPO1, ZBTB38, Wnt/3-Catenin pathway, NFκB pathway, DNA repair pathway, MAPK pathway and/or ubiquitination pathway.

A further embodiment of the present invention is the use of compound 9, or another LMP7-selective inhibitor described herein, to treat subjects with multiple myeloma, MGUS, SMM or other malignancies that show an incomplete response to therapies such as pan-proteasome inhibitors (e.g. bortezomib, carfilzomib, ixazomib), as demonstrated by assays that assess pharmacodynamic biomarkers including LMP7 activity, tumor apoptosis (e.g. Caspase activity) or other standard methodologies uses to assess the response of multiple myeloma patients to therapy such as immunoglobulin, free light chain, M protein, MRD, histology (e.g. IHC, in situ hybridization), imaging (e.g. PET/CT, MRI) and/or flow cytometry.

Another embodiment of the present invention is the use of compound 9, or another LMP7-selective inhibitor described herein, to treat subjects with plasma cell leukemia, solitary plasmacytoma, lymphoplasmacytic lymphoma, amyloid light-chain (AL) amyloidosis or Waldenström's macroglobulinemia (WM) that show an incomplete response to therapies such as pan-proteasome inhibitors (e.g. bortezomib, carfilzomib, ixazomib).

Another embodiment of the present invention is the use of compound 9, or another LMP7-selective inhibitor described herein, to treat subjects with solid tumors that show an incomplete response to therapies such as pan-proteasome inhibitors (e.g. bortezomib, carfilzomib, ixazomib).

A further embodiment of the present invention is the use of compound 9, or another LMP7-selective inhibitor described herein, administered in combination with EGFR pathway inhibitors (e.g. erlotinib, afatinib, gefitinib, cetuximab, panitumumab, lapatinib, osimertinib, trastuzumab, pertuzumab) in subjects with multiple myeloma, MGUS, SMM or other malignancies that are positive for EGFR genetic alteration.

A further embodiment of the present invention is the use of compound 9, or another LMP7-selective inhibitor described herein, administered in combination with EGFR pathway inhibitors (e.g. erlotinib, afatinib, gefitinib, cetuximab, panitumumab, lapatinib, osimertinib, trastuzumab, pertuzumab) in subjects with plasma cell leukemia, solitary plasmacytoma, lymphoplasmacytic lymphoma, amyloid light-chain (AL) amyloidosis or Waldenström's macroglobulinemia (WM) that are positive for EGFR genetic alteration.

A further embodiment of the present invention is the use of compound 9, or another LMP7-selective inhibitor described herein, administered in combination with EGFR pathway inhibitors (e.g. erlotinib, afatinib, gefitinib, cetuximab, panitumumab, lapatinib, osimertinib, trastuzumab, pertuzumab) in subjects with solid tumors that are positive for EGFR genetic alteration.

One additional embodiment of the invention is the use of compound 9, or another LMP7-selective inhibitor described herein, administered in combination with MAPK pathway inhibitors (e.g. trametinib, cobimetinib, binimetinib, selumetinib, refametinib, pimasertib, AMG 510, MRTX849, vemurafenib, dabrafenib, encorafenib, LXH254, HM95573, XL281, RAF265, RAF709, LY3009120, ulixertinib, SCH772984, TNO155, RMC-4630, JAB-3068, JAB-3312, AMG-510, MRTX849, LY3499446 and/or BI 1701963 in subjects with multiple myeloma, MGUS, SMM or other malignancies that are positive for MAPK pathway genetic alterations in KRAS, NRAS, BRAF, HRAS, MAP4K3, and/or NF1.

One additional embodiment of the invention is the use of compound 9, or another LMP7-selective inhibitor described herein, administered in combination with MAPK pathway inhibitors (e.g. trametinib, cobimetinib, binimetinib, selumetinib, refametinib, pimasertib, AMG 510, MRTX849, vemurafenib, dabrafenib, encorafenib, LXH254, HM95573, XL281, RAF265, RAF709, LY3009120, ulixertinib, SCH772984, TNO155, RMC-4630, JAB-3068, JAB-3312, AMG-510, MRTX849, LY3499446 and/or BI 1701963 in subjects with plasma cell leukemia, solitary plasmacytoma, lymphoplasmacytic lymphoma, amyloid light-chain (AL) amyloidosis or Waldenström's macroglobulinemia (WM) that are positive for MAPK pathway genetic alterations in KRAS, NRAS, BRAF, HRAS, MAP4K3, and/or NF1.

One additional embodiment of the invention is the use of compound 9, or another LMP7-selective inhibitor described herein, administered in combination with MAPK pathway inhibitors (e.g. trametinib, cobimetinib, binimetinib, selumetinib, refametinib, pimasertib, AMG 510, MRTX849, vemurafenib, dabrafenib, encorafenib, LXH254, HM95573, XL281, RAF265, RAF709, LY3009120, ulixertinib, SCH772984, TNO155, RMC-4630, JAB-3068, JAB-3312, AMG-510, MRTX849, LY3499446 and/or BI 1701963 in subjects with solid tumors that are positive for MAPK pathway genetic alterations in KRAS, NRAS, BRAF, HRAS, MAP4K3, and/or NF1.

An additional embodiment of the invention is the use of compound 9, or another LMP7-selective inhibitor described herein, administered in combination with XPO1 inhibitors (e.g. selinexor, KPT-8602) in patients with multiple myeloma, MGUS, SMM or other malignancies that are positive for XPO1 genetic alterations.

An additional embodiment of the invention is the use of compound 9, or another LMP7-selective inhibitor described herein, administered in combination with XPO1 inhibitors (e.g. selinexor, KPT-8602) in patients with plasma cell leukemia, solitary plasmacytoma, lymphoplasmacytic lymphoma, amyloid light-chain (AL) amyloidosis or Waldenström's macroglobulinemia (WM) that are positive for XPO1 genetic alterations.

An additional embodiment of the invention is the use of compound 9, or another LMP7-selective inhibitor described herein, administered in combination with XPO1 inhibitors (e.g. selinexor, KPT-8602) in patients with solid tumors that are positive for XPO1 genetic alterations.

One embodiment of the invention is the use of compound 9, or another LMP7-selective inhibitor described herein, administered in combination with DNA repair pathway inhibitors (e.g. talazoparib, niraparib, olaparib, veliparib, rucaparib, pamiparib, AZD7648, M3814, CC-115, BAY1895344, AZD6738, M6620, M4344, M1774, M4076, M3541, AZD0157, AZD1390, prexasertib, GDC-0425, SRA-737, AZD1775 and/or Debio 0123) in patients with multiple myeloma, MGUS, SMM or other malignancies that are positive for DNA repair pathway genetic alterations (e.g. BRCA1, BRCA2, ATM, ATR, TP53).

One embodiment of the invention is the use of compound 9, or another LMP7-selective inhibitor described herein, administered in combination with DNA repair pathway inhibitors (e.g. talazoparib, niraparib, olaparib, veliparib, rucaparib, pamiparib, AZD7648, M3814, CC-115, BAY1895344, AZD6738, M6620, M4344, M1774, M4076, M3541, AZD0157, AZD1390, prexasertib, GDC-0425, SRA-737, AZD1775 and/or Debio 0123) in patients with plasma cell leukemia, solitary plasmacytoma, lymphoplasmacytic lymphoma, amyloid light-chain (AL) amyloidosis or Waldenström's macroglobulinemia (WM) that are positive for DNA repair pathway genetic alterations (e.g. BRCA1, BRCA2, ATM, ATR, TP53).

One embodiment of the invention is the use of compound 9, or another LMP7-selective inhibitor described herein, administered in combination with DNA repair pathway inhibitors (e.g. talazoparib, niraparib, olaparib, veliparib, rucaparib, pamiparib, AZD7648, M3814, CC-115, BAY1895344, AZD6738, M6620, M4344, M1774, M4076, M3541, AZD0157, AZD1390, prexasertib, GDC-0425, SRA-737, AZD1775 and/or Debio 0123) in patients with solid tumors that are positive for DNA repair pathway genetic alterations (e.g. BRCA1, BRCA2, ATM, ATR, TP53).

A further embodiment of the present invention is the use of compound 9, or another LMP7-selective inhibitor described herein, administered in combination with FGFR pathway inhibitors (e.g. erdafitinib, AZD4547, LY2874455, Debio 1347, NVP-BGJ398, pemigatinib, rogaratinib, PRN1371, TAS-120, nintedanib) in patients with multiple myeloma, MGUS, SMM or other malignancies that are positive for the high-risk t(4;14) cytogenetic abnormality/translocation and/or for FGFR3 genetic alterations.

A further embodiment of the present invention is the use of compound 9, or another LMP7-selective inhibitor described herein, administered in combination with FGFR pathway inhibitors (e.g. erdafitinib, AZD4547, LY2874455, Debio 1347, NVP-BGJ398, pemigatinib, rogaratinib, PRN1371, TAS-120, nintedanib) in patients with plasma cell leukemia, solitary plasmacytoma, lymphoplasmacytic lymphoma, amyloid light-chain (AL) amyloidosis or Waldenström's macroglobulinemia (WM) that are positive for the high-risk t(4;14) cytogenetic abnormality/translocation and/or for FGFR3 genetic alterations.

A further embodiment of the present invention is the use of compound 9, or another LMP7-selective inhibitor described herein, administered in combination with FGFR pathway inhibitors (e.g. erdafitinib, AZD4547, LY2874455, Debio 1347, NVP-BGJ398, pemigatinib, rogaratinib, PRN1371, TAS-120, nintedanib) in patients with solid tumors that are positive for the high-risk t(4;14) cytogenetic abnormality/translocation and/or for FGFR3 genetic alterations.

An additional embodiment of the invention is the use of compound 9, or another LMP7-selective inhibitor described herein, administered in combination with PI3K/AKT/mTOR pathway inhibitors (e.g. rapamycin, temsirolimus, everolimus, ridaforolimus, alpelisib, idelalisib, copanlisib, duvelisib, MK-2206, AZD5363) in patients with multiple myeloma, MGUS, SMM or other malignancies that are positive for PI3K/AKT/mTOR pathway genetic alterations (e.g. RICTOR, RAPTOR, PIK3CA, PIK3R1, PTEN, AKT, IRS1, IGF1R).

An additional embodiment of the invention is the use of compound 9, or another LMP7-selective inhibitor described herein, administered in combination with PI3K/AKT/mTOR pathway inhibitors (e.g. rapamycin, temsirolimus, everolimus, ridaforolimus, alpelisib, idelalisib, copanlisib, duvelisib, MK-2206, AZD5363) in patients with plasma cell leukemia, solitary plasmacytoma, lymphoplasmacytic lymphoma, amyloid light-chain (AL) amyloidosis or Waldenström's macroglobulinemia (WM) that are positive for PI3K/AKT/mTOR pathway genetic alterations (e.g. RICTOR, RAPTOR, PIK3CA, PIK3R1, PTEN, AKT, IRS1, IGF1R).

An additional embodiment of the invention is the use of compound 9, or another LMP7-selective inhibitor described herein, administered in combination with PI3K/AKT/mTOR pathway inhibitors (e.g. rapamycin, temsirolimus, everolimus, ridaforolimus, alpelisib, idelalisib, copanlisib, duvelisib, MK-2206, AZD5363) in patients with solid tumors that are positive for PI3K/AKT/mTOR pathway genetic alterations (e.g. RICTOR, RAPTOR, PIK3CA, PIK3R1, PTEN, AKT, IRS1, IGF1R).

A further embodiment of the present invention is the use of compound 9, or another LMP7-selective inhibitor described herein, administered in combination with MCL1 inhibitors or apoptosis modulators (e.g. A-1210477, VU661013, AZD5991, AMG-176, AMG-397, S63845, S64315, venetoclax, HDM201, NVP-CGM097, RG-7112, MK-8242, RG-7388, SAR405838, AMG-232, DS-3032, RG7775, APG-115) in patients with multiple myeloma, MGUS, SMM or other malignancies that are positive for MCL1 or apoptosis modulator pathway genetic alterations (e.g. BCL2, BCLXL, TP53).

A further embodiment of the present invention is the use of compound 9, or another LMP7-selective inhibitor described herein, administered in combination with MCL1 inhibitors or apoptosis modulators (e.g. A-1210477, VU661013, AZD5991, AMG-176, AMG-397, S63845, S64315, venetoclax, HDM201, NVP-CGM097, RG-7112, MK-8242, RG-7388, SAR405838, AMG-232, DS-3032, RG7775, APG-115) in patients with plasma cell leukemia, solitary plasmacytoma, lymphoplasmacytic lymphoma, amyloid light-chain (AL) amyloidosis or Waldenström's macroglobulinemia (WM) that are positive for MCL1 or apoptosis modulator pathway genetic alterations (e.g. BCL2, BCLXL, TP53).

A further embodiment of the present invention is the use of compound 9, or another LMP7-selective inhibitor described herein, administered in combination with MCL1 inhibitors or apoptosis modulators (e.g. A-1210477, VU661013, AZD5991, AMG-176, AMG-397, S63845, S64315, venetoclax, HDM201, NVP-CGM097, RG-7112, MK-8242, RG-7388, SAR405838, AMG-232, DS-3032, RG7775, APG-115) in patients with solid tumors that are positive for MCL1 or apoptosis modulator pathway genetic alterations (e.g. BCL2, BCLXL, TP53).

### Definitions

“Pan-proteasome inhibitor” as used herein is defined as an approved or experimental compound which inhibits subunits of the immunoproteasome and constitutive proteasome. Examples of pan-proteasome inhibitors are bortezomib, carfilzomib, ixazomib, oprozomib, marizomib and delanzomib.

“Genetic alteration” as used herein is defined as genetic mutation, deregulated expression, or dependency. The assessment of the genetic alterations and cytogenetic abnormalities/translocations described above can be performed by nucleotide sequencing, karyotyping, FISH, protein and/or RNA expression analyses, flow cytometry and/or other standard methodologies.

The expression “effective amount” denotes the amount of a medicament or of a pharmaceutical active ingredient which causes in a tissue, system, animal or human a biological or medical response which is sought or desired, for example, by a researcher or physician. The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically acceptable excipient(s) and/or carrier(s) utilized, and similar factors within the knowledge and expertise of the attending physician.

In addition, the expression “therapeutically effective amount” denotes an amount which, compared with a corresponding subject who has not received this amount, has the following consequence: improved treatment, healing, elimination of a disease, syndrome, condition, complaint, disorder or side-effects or also the reduction in the advance of a disease, complaint or disorder. The expression “therapeutically effective amount” also encompasses the amounts which are effective for increasing normal physiological function. With respect to treatment of cancer and/or blood disorders, “therapeutically effective amount” also encompasses an amount which leads to the remission of disease (even if only temporary), decrease in the tumor burden of a subject, a delay in the progression of the disease, a delay or reduction of metastases, extension of overall survival of the subject, and/or amelioration of one or more symptoms of disease.

Compounds of the Invention

TABLE 1
List of exemplary compounds

| Compound | | |
|---|---|---|
| No. | Structure | Name |
| 1 | 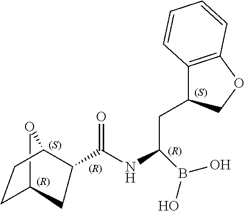 | [(1R)-2-[(3S)-2,3-dihydro-1- benzofuran-3-yl]-1- {[(1S,2R,4R)-7- oxabicyclo[2.2.1]heptan-2- yl]formamido}ethyl]boronic acid |
| 2 | 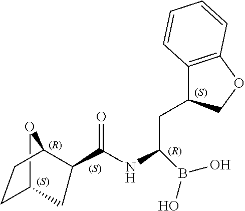 | [(1R)-2-[(3S)-2,3-dihydro-1- benzofuran-3-yl]-1- {[(1R,2S,4S)-7- oxabicyclo[2.2.1]heptan-2- yl]formamido}ethyl]boronic acid |
| 3 | 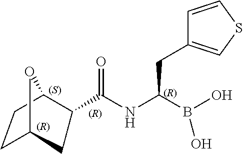 | [(1R)-1-{[(1S,2R,4R)-7- oxabicyclo[2.2.1]heptan-2- yl]formamido}-2-(thiophen-3- yl)ethyl]boronic acid |
| 4 | 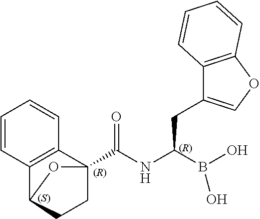 | [(1R)-2-(1-benzofuran-3-yl)-1- {[(1R,8S)-11- oxatricyclo[6.2.1.0²,⁷]undeca- 2(7),3,5-trien-1- yl]formamido}ethyl]boronic acid |
| 5 | 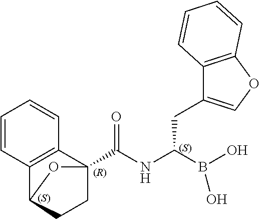 | [(1S)-2-(1-benzofuran-3-yl)-1- {[(1R,8S)-11- oxatricyclo[6.2.1.0²,⁷]undeca- 2(7),3,5-trien-1- yl]formamido}ethyl]boronic acid |
| 6 | 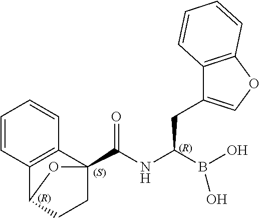 | [(1R)-2-(1-benzofuran-3-yl)-1- {[(1S,8R)-11- oxatricyclo[6.2.1.0²,⁷]undeca- 2(7),3,5-trien-1- yl]formamido}ethyl]boronic acid |
| 7 | 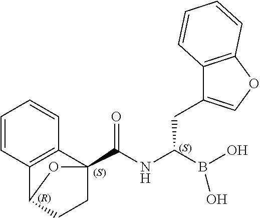 | [(1S)-2-(1-benzofuran-3-yl)-1- {[(1S,8R)-11- oxatricyclo[6.2.1.0²,⁷]undeca- 2(7),3,5-trien-1- yl]formamido}ethyl]boronic acid |
| 8 | 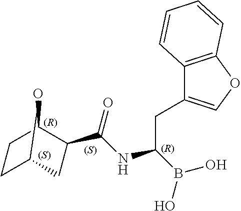 | [(1R)-2-(1-benzofuran-3-yl)-1- {[(1R,2S,4S)-7- oxabicyclo[2.2.1]heptan-2- yl]formamido}ethyl]boronic acid |
| 9 | 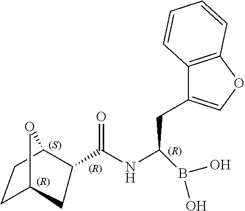 | [(1R)-2-(1-benzofuran-3-yl)-1- {[(1S,2R,4R)-7- oxabicyclo[2.2.1]heptan-2- yl]formamido}ethyl]boronic acid |
| 10 | 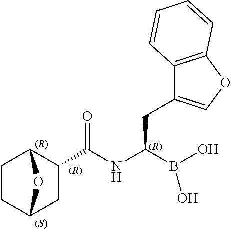 | [(1R)-2-(1-benzofuran-3-yl)-1- {[(1R,2R,4S)-7- oxabicyclo[2.2.1]heptan-2- yl]formamido}ethyl]boronic acid |
| 11 | 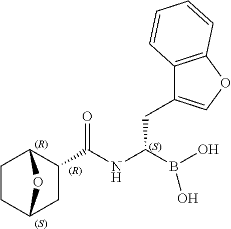 | [(1S)-2-(1-benzofuran-3-yl)-1- {[(1R,2R,4S)-7- oxabicyclo[2.2.1]heptan-2- yl]formamido}ethyl]boronic acid |
| 12 | 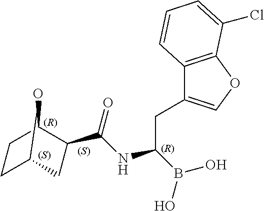 | [(1R)-2-(7-chloro-1- benzofuran-3-yl)-1- {[(1R,2S,4S)-7- oxabicyclo[2.2.1]heptan-2- yl]formamido}ethyl]boronic acid |
| 13 | 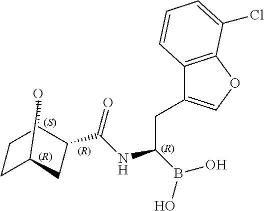 | [(1R)-2-(7-chloro-1- benzofuran-3-yl)-1- {[(1S,4R,4R)-7- oxabicyclo[2.2.1]heptan-2- yl]formamido}ethyl]boronic acid |
| 14 | 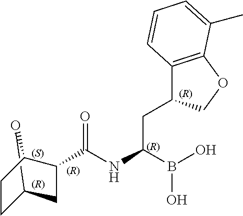 | [(1R)-2-[(3R)-7-methyl-2,3- dihydro-1-benzofuran-3-yl]-1- {[(1S,4R,4R)-7- oxabicyclo[2.2.1]heptan-2- yl]formamido}ethyl]boronic acid |
| 15 | 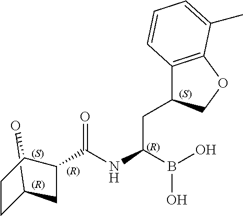 | [(1R)-2-[(3S)-7-methyl-2,3- dihydro-1-benzofuran-3-yl]-1- {[(1S,4R,4R)-7- oxabicyclo[2.2.1]heptan-2- yl]formamido}ethyl]boronic acid |
| 16 | 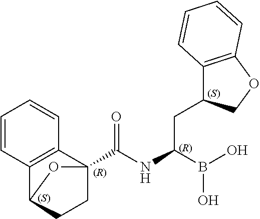 | [(1R)-2-[(3S)-2,3-dihydro-1- benzofuran-3-yl]-1-{[(1R,8S)- 11- oxatricyclo[6.2.1.0²,⁷]undeca- 2(7),3,5-trien-1- yl]formamido}ethyl]boronic acid |
| 17 | 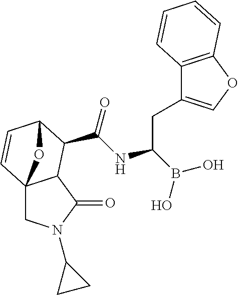 [SMILES: O=C(N[C@@H](Cc1coc2ccccc12)B(O)O)[C@H]1C2C(=O)N(C3CC3)C[C@]23C=C[C@H]1O3] | [(1R)-2-(1-benzofuran-3-yl)-1- {[(1S,6S,7R)-3-cyclopropyl-4- oxo-10-oxa-3- azatricyclo[5.2.10¹,⁵]dec-8-en- 6-yl]formamido}ethyl]boronic acid |
| 18 | 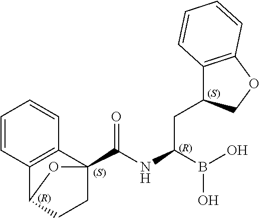 | [(1R)-2-[(3S)-2,3-dihydro-1- benzofuran-3-yl]-1-{[(1S,8R)- 11- oxatricyclo[6.2.1.0²,⁷]undeca- 2(7),3,5-trien-1- yl]formamido}ethyl]boronic acid |
| 19 | 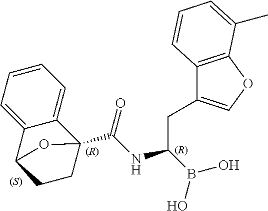 | [(1R)-2-(7-methyl-1- benzofuran-3-yl)-1-{[(1R,8S)- 11- oxatricyclo[6.2.1.0²,⁷]undeca- 2,4,6-trien-1- yl]formamido}ethyl]boronic acid |
| 20 | 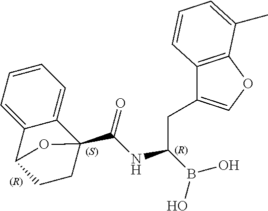 | [(1R)-2-(7-methyl-1- benzofuran-3-yl)-1-{[(1S,8R)- 11- oxatricyclo[6.2.1.0²,⁷]undeca- 2,4,6-trien-1- yl]formamido}ethyl]boronic acid |
| 21 | 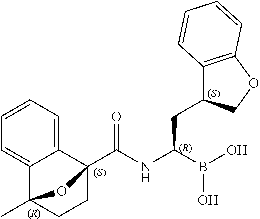 | [(1R)-2-[(3S)-2,3-dihydro-1- benzofuran-3-yl]-1-{[(1S,8R)- 8-methyl-11- oxatricyclo[6.2.1.0²,⁷]undeca- 2,4,6-trien-1- yl]formamido}ethyl]boronic acid |
| 22 | 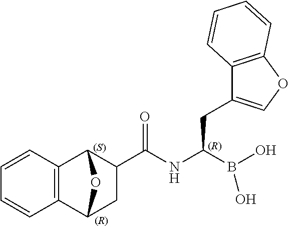 | [(1R)-2-(1-benzofuran-3-yl)-1- {[(1R,8S)-11- oxatricyclo[6.2.1.0²,⁷]undeca- 2(7),3,5-trien-9- yl]formamido}ethyl]boronic acid |
| 23 | 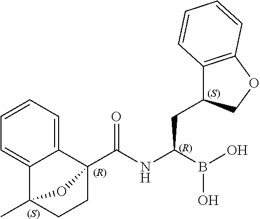 | [(1R)-2-[(3S)-2,3-dihydro-1- benzofuran-3-yl]-1-{[(1R,8S)- 8-methyl-11- oxatricyclo[6.2.1.0²,⁷]undeca- 2,4,6-trien-1- yl]formamido}ethyl]boronic acid |
| 24 | 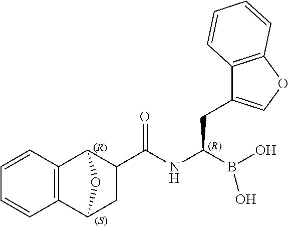 | [(1R)-2-(1-benzofuran-3-yl)-1- {[(1S,8R)-11- oxatricyclo[6.2.1.0²,⁷]undeca- 2(7),3,5-trien-9- yl]formamido}ethyl]boronic acid |
| 25 | 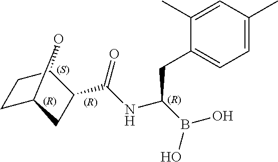 | [(1R)-2-(2,4-dimethylphenyl)- 1-{[(1S,2R,4R)-7- oxabicyclo[2.2.1]heptan-2- yl]formamido}ethyl]boronic acid |
| 26 | 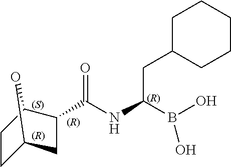 | [(1R)-2-cyclohexyl-1- {[(1S,2R,4R)-7- oxabicyclo[2.2.1]heptan-2- yl]formamido}ethyl]boronic acid |
| 27 | 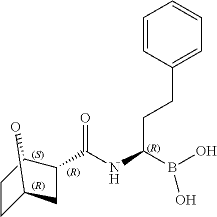 | [(1R)-1-{[(1S,2R,4R)-7- oxabicyclo[2.2.1]heptan-2- yl]formamido}-3- phenylpropyl]boronic acid |
| 28 | 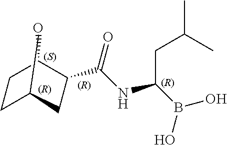 | [(1R)-3-methyl-1- {[(1S,2R,4R)-7- oxabicyclo[2.2.1]heptan-2- yl]formamido}butyl]boronic acid |

In one embodiment, the compound of the invention is compound 9.

Mechanism of action analyses revealed that compound 9 has a more pronounced and longer inhibition of the enzymatic function of LMP7 and longer induction of apoptosis in multiple myeloma tumor cells in vivo as compared to that observed with the pan-proteasome inhibitors bortezomib, carfilzomib and ixazomib. These findings indicate that multiple myeloma patients in which suboptimal suppression of LMP7, induction of tumor cell apoptosis, or modulation of other pharmacodynamic biomarkers of relevance to multiple myeloma (e.g. immunoglobulin, free light chain, M protein, minimal residual disease (MRD), histology, imaging, flow cytometry) are observed upon treatment with therapies such as pan-proteasome inhibitors (e.g. bortezomib, carfilzomib, ixazomib) could derive a therapeutic benefit from treatment with compound 9, or other LMP7-selective inhibitors described herein.

Pharmaceutical Formulations/Dosage

Pharmaceutical formulations can be administered in the form of dosage units, which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process, which is generally known in the pharmaceutical art.

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or a sugar alcohol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, a stearic salt or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, waxes, and the like. The lubricants used in these dosage forms include sodium oleate, stearic salts, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, an alginate or gelatine, a dissolution retardant, such as, for example, paraffin, an absorption accelerator, such as, for example, a quaternary salt, and/or an absorbant, such as, for example, bentonite or kaolin. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The active ingredients can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present Dyes can be added to these coatings in order to be able to differentiate between different dosage units.

The compositions/formulations according to the invention can be used as medicaments in human and veterinary medicine.

A therapeutically effective amount of a compound of the invention and of the other active ingredient depends on a number of factors, including, for example, the age and weight of the animal, the precise disease condition which requires treatment, and its severity, the nature of the formulation and the method of administration, and is ultimately determined by the treating doctor or vet. However, an effective amount of a compound is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a pharmaceutically acceptable salt or solvate thereof can be determined as the fraction of the effective amount of the compound per se.

Combination Administration

When an LMP7-selective inhibitor of the invention is administered in combination with one or more additional therapeutic agents, the two or more compounds may be administered concurrently, consecutively, and/or on independent administration schedules. One embodiment of the invention provides for the use of an LMP7-selective inhibitor of the invention in combination with one or more additional therapeutic agents to treat a blood disorder and/or cancer, wherein each active ingredient is administered on an independent schedule, but the subject is administered at least two agents during the course of treatment.

In one embodiment, the invention provides for the use of an LMP7-selective inhibitor of the invention in combination with one or more additional therapeutic agents to treat a blood disorder and/or cancer, wherein each active ingredient is administered consecutively. In one aspect of this embodiment, consecutive administration comprises administering at least one dose of the at least two active agents to a subject in need thereof within a week of each other. In another aspect of this embodiment, consecutive administration comprises administering at least one does of the at least two active ingredients to a subject in need thereof within 48 hours of each other. In a further aspect of this embodiment, consecutive administration comprises administering at least one does of the at least two active ingredients to a subject in need thereof within 24 hours of each other. In another aspect of this embodiment, consecutive administration comprises administering at least one does of the at least two active ingredients to a subject in need thereof within 12 hours of each other.

In one embodiment, the invention provides for the use of an LMP7-selective inhibitor of the invention in combination with one or more additional therapeutic agents to treat a blood disorder and/or cancer, wherein each active ingredient is administered concurrently. In one aspect of this embodiment, concurrent administration comprises administering at least one dose of the at least two active agents to a subject in need thereof within about an hour of each other.

Kits

The present invention further relates to a set (kit) consisting of separate packs of
(a) an effective amount of a compound of the formula (I) and/or a prodrug, solvate, tautomer, oligomer, adduct or stereoisomer thereof as well as a pharmaceutically acceptable salt of each of the foregoing, including mixtures thereof in all ratios, and
(b) an effective amount of a further medicament active ingredient.

The compounds of the present invention can be prepared according to the procedures described in PCT Application No. WO 19/38250, which included in its entirety herein by reference.

### EXAMPLES

### Example 1: Efficacy of Bortezomib, Carfildzomib and Ixazomib and Compound 9 in the Multiple Myeloma XenoQraft Model OPM-2

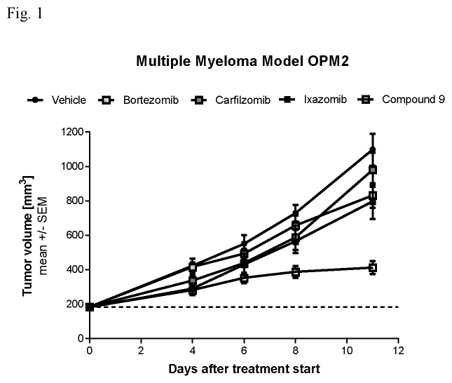

The human multiple myeloma cell line OPM-2 was obtained from the German Collection of Microorganisms and Cell Cultures GmbH (DSMZ). 100 μL of a suspension of 5 million cells in phosphate-buffered saline (PBS) mixed 1:1 with Matrigel (Becton Dickinson) was injected per mouse. Bortezomib was formulated in Mannitol (Merck KGaA) in 0.9% NaCl and applied intravenously (i.v.) to mice at a dose of 0.5 mg/kg twice per week. Ixazomib was formulated in 5% KLEPTOSE® in water and applied orally (per os) to mice at a dose of 3 mg/kg twice per week. Carfilzomib was formulated in 5% KLEPTOSE® (AppliChem) in 50 mM sodium citrate buffer and applied by intraperitoneal (i.p.) injection to mice at a dose of 2 mg/kg twice per week. Compound 9 was formulated in 0.5% METHOCEL™ Premium K4M (Colorcon) and 0.25% Tween® 20 in PBS at applied per os to mice once daily at a dose of 10 mg/kg. Mean tumor volume and standard error of the mean (SEM) are indicated. Results are shown in FIG. 1. Compound 9 shows increased efficacy as compared to bortezomib, carfilzomib, and ixazomib.

### Example 2: Efficacy of Bortezomib, Cardzomib and Compound 9 in the Multiple Myeloma Xenograft Model NCI-H929

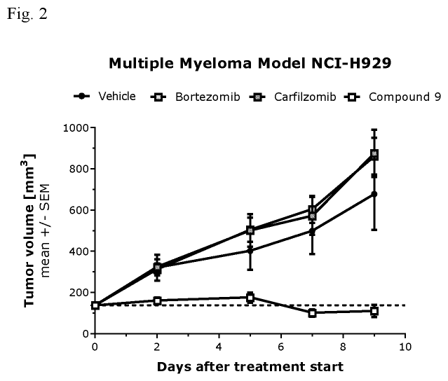

The human multiple myeloma cell line NCI-H929 was obtained from the American Type Culture Collection (ATCC). 100 μL of a suspension of 5 million cells in PBS mixed 1:1 with Matrigel was injected per mouse. Bortezomib, carfilzomib and compound 9 were formulated and applied as described in Example 1. Mean tumor volume and SEM are indicated. Results are shown in FIG. 2. Compound 9 shows increased efficacy as compared to bortezomib and carfilzomib.

### Example 3: Efficacy of Compound 9 and Ixazomib in the Multiple Myeloma Xenograft Model NCI-H929

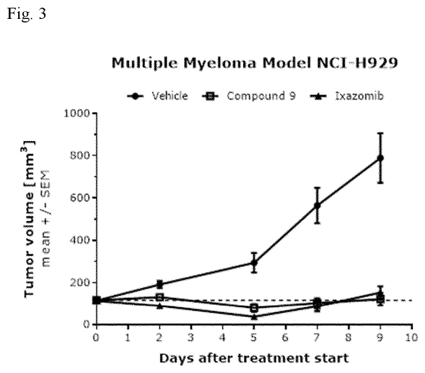

NCI-H929 xenograft tumors were established as described in Example 2. Compound 9 and ixazomib were formulated and applied as described in Example 1. Mean tumor volume and SEM are indicated in FIG. 3.

### Example 4: Efficacy of Bortezomib, Carfilzomib, Ixazomib and Compound 9 in the Multiple Myeloma Xenograft Model MM.1S

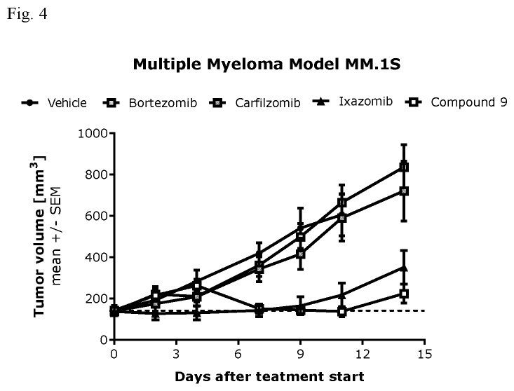

The human multiple myeloma cell line MM.1S was obtained from the ATCC. 100 μL of a suspension of 5 million cells in PBS was injected per mouse. Bortezomib, ixazomib and compound 9 were formulated and applied as described in Example 1. Carfilzomib was formulated as described in Example 1 and applied intravenously (i.v.) to mice at a dose of 3 mg/kg twice per week. Mean tumor volume and SEM are indicated in FIG. 4. FIG. 4 shows an increased anti-tumor activity of compound 9 as compared to bortezomib and carfilzomib, and comparable activity as compared to ixazomib.

### Example 5: Efficacy of Bortezomib, Carfilzomib, Ixazomib and Compound 9 in the Multiple Myeloma Xenograft Model RPMI 8826

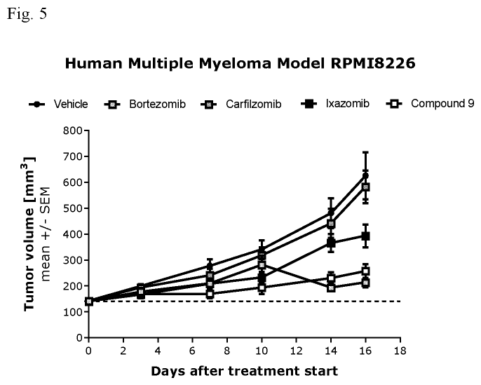

The human multiple myeloma cell line RPMI 8826 was obtained from the ATCC. 100 μL of a suspension of 5 million cells in PBS mixed 1:1 with Matrigel was injected per mouse. Bortezomib, carfilzomib, ixazomib and compound 9 were formulated and applied as described in Example 1. Mean tumor volume and SEM are indicated in FIG. 5. FIG. 5 shows the increased in vivo activity of compound 9 as compared to ixazomib and carfilzomib, and comparable activity when compared to bortezomib.

### Example 6: Effect of Compounds 9, Bortezomib, Carfilzomib and Ixazomib on LMP7 Activity in MM.1S Xenograft Tumors In Vivo

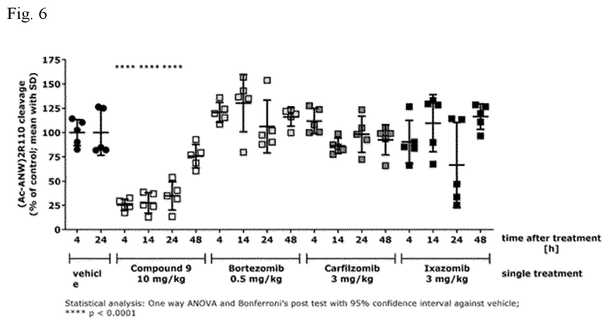

MM.1S xenograft tumors were established as described in Example 4. Compound 9, bortezomib, carfilzomib and ixazomib were formulated as described in Example 1. Compound 9 was applied once per os to mice at a dose of 10 mg/kg. Bortezomib was applied once i.v. to mice at a dose of 0.5 mg/kg. Ixazomib was applied once per os to mice at a dose of 3 mg/kg. Carfilzomib was applied once i.v. at a dose of 3 mg/kg. MM1S tumors were collected immediately following mouse euthanasia and lysed as described previously (Buchstaller et al., 2019). For assessment of LMP7 activity, 10 μg of tumor lysate protein in a total volume of 50 μL was mixed in 96-well plates with 50 μl of a buffer containing 100 mM HEPES pH 7.6, 60 mM MgSO₄, 1 mM EDTA, 40 μg/ml digitonin and the fluorogenic LMP7 substrate (Ac-ANW)2R110 (from Biomol) at a final concentration of 10 μM. Plates were then shaken briefly, incubated for 60 min at 37° C. and then centrifuged at 300×g. Fluorescence (excitation 485 nm, emission 535 nm) was measured using an EnVision 2104 plate reader (PerkinElmer). Mean and standard deviation (SD) LMP7 activity values (% control) are indicated in FIG. 6. FIG. 6 shows the effect of compound 9 on LMP7 activity was stronger and more prolonged than for the other pan-proteasome inhibitors tested.

### Example 7: Effect of Compound 9. Bortezomib, Carfilzomib and Ixazomib on Caspase 3/7 Activity in MM.1S Xenograft Tumors In Vivo

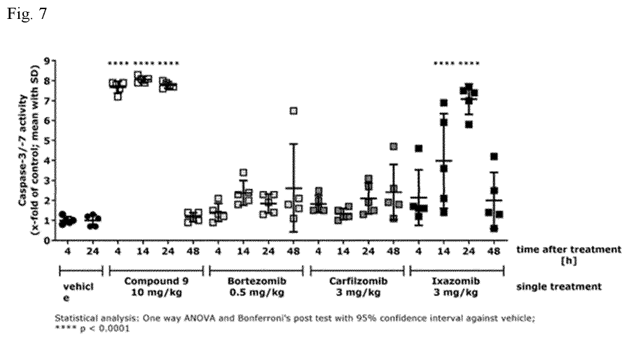

MM.1S xenograft tumors samples from the same experiment described in Example 6 were used for assessment of Caspase-3/-7 activity as an indication of tumor cell apoptosis. 50 μg of tumor lysate protein in a total volume of 50 μL was mixed with the Caspase-Glo®3/7 Reagent (Promega) in 96-well plates according to the manufacturer's instruction. Luminescence was measured using an Envision 2104 plate reader (PerkinElmer). The mean and SD for the fold increase in Caspase-3/-7 activity compared to vehicle control tumors is indicated in FIG. 7. FIG. 7 clearly shows that the induction of apoptosis by compound 9 was longer than compared to the pan-proteasome inhibitors.

### REFERENCES

Ahmed, A. S., Li, J., Erlandsson-Harris, H., Stark, A., Bakalkin, G., and Ahmed, M. (2012). Suppression of pain and joint destruction by inhibition of the proteasome system in experimental osteoarthritis. Pain 153, 18-26.
Allen, S., Heath, P. R., Kirby, J., Wharton, S. B., Cookson, M. R, Menzies, F. M., Banks, R E., and Shaw, P. J. (2003). Analysis of the cytosolic proteome in a cell culture model of familial amyotrophic lateral sclerosis reveals alterations to the proteasome, antioxidant defenses, and nitric oxide synthetic pathways. J Biol Chem 278, 6371-6383.
Altun, M., Galardy, P. J., Shringarpure, R, Hideshima, T., LeBlanc, R, Anderson, K. C., Ploegh, H. L., and Kessler, B. M. (2005). Effects of PS-341 on the activity and composition of proteasomes in multiple myeloma cells. Cancer Res 65, 7896-7901.
Anan, A., Baskin-Bey, E. S., Isomoto, H., Mott, J. L., Bronk, S. F., Albrecht, J. H., and Gores, G. J. (2006). Proteasome inhibition attenuates hepatic injury in the bile duct-ligated mouse. Am J Physiol Gastrointest Liver Physiol 291, G709-716.
Arastu-Kapur, S., Anderl, J. L., Kraus, M., Parlati, F., Shenk, K. D., Lee, S. J., Muchamuel, T., Bennett, M. K., Driessen, C., Ball, A. J., et al. (2011). Nonproteasomal targets of the proteasome inhibitors bortezomib and carfilzomib: a link to clinical adverse events. Clin Cancer Res 17, 2734-2743.
Assouline, S. E., Chang, J., Cheson, B. D., Rifkin, R, Hamburg, S., Reyes, R., Hui, A. M., Yu, J., Gupta, N., Di Bacco, A., et al. (2014). Phase 1 dose-escalation study of IV ixazomib, an investigational proteasome inhibitor, in patients with relapsed/refractory lymphoma. Blood Cancer J 4, e251.
Basler, M., Dajee, M., Moll, C., Groettrup, M., and Kirk, C. J. (2010). Prevention of experimental colitis by a selective inhibitor of the immunoproteasome. J Immunol 185, 634-641.
Bontscho, J., Schreiber, A., Manz, R. A., Schneider, W., Luft, F. C., and Kettritz, R. (2011). Myeloperoxidase-specific plasma cell depletion by bortezomib protects from anti-neutrophil cytoplasmic autoantibodies-induced glomerulonephritis. J Am Soc Nephrol 22, 336-348.
Buchstaller, H. P., Anlauf, U., Dorsch, D., Kuhn, D., Lehmann, M., Leuthner, B., Musil, D., Radtki, D., Ritzert, C., Rohdich, F., et al. (2019). Discovery and Optimization of 2-Arylquinazolin-4-ones into a Potent and Selective Tankyrase Inhibitor Modulating Wnt Pathway Activity. J Med Chem 62, 7897-7909.
Bustoros, M., Mouhieddine, T. H., Detappe, A., and Ghobrial, I. M. (2017). Established and Novel Prognostic Biomarkers in Multiple Myeloma. Am Soc Clin Oncol Educ Book 37, 548-560.
Chanukuppa, V., Paul, D., Taunk, K., Chatterjee, T., Sharma, S., Kumar, S., Santra, M. K., and Rapole, S. (2019). XPO1 is a critical player for bortezomib resistance in multiple myeloma: A quantitative proteomic approach. J Proteomics 209, 103504.
Chen, D., Frezza, M., Schmitt, S., Kanwar, J., and Dou, Q. P. (2011). Bortezomib as the first proteasome inhibitor anticancer drug: current status and future perspectives. Curr Cancer Drug Targets 11, 239-253.
Chong, K. Y., Hsu, C. J., Hung, T. H., Hu, H. S., Huang, T. T., Wang, T. H., Wang, C., Chen, C. M., Choo, K. B., and Tseng, C. P. (2015). Wnt pathway activation and ABCB1 expression account for attenuation of proteasome inhibitor-mediated apoptosis in multidrug-resistant cancer cells. Cancer Biol Ther 16, 149-159.
Coppo, R, Camilla, R, Alfarano, A., Balegno, S., Mancuso, D., Peruzzi, L., Amore, A., Dal Canton, A., Sepe, V., and Tovo, P. (2009). Upregulation of the immunoproteasome in peripheral blood mononuclear cells of patients with IgA nephropathy. Kidney Int 75, 536-541.
Egerer, T., Martinez-Gamboa, L., Dankof, A., Stuhlmuller, B., Dorner, T., Krenn, V., Egerer, K., Rudolph, P. E., Burmester, G. R, and Feist, E. (2006). Tissue-specific up-regulation of the proteasome subunit beta5i (LMP7) in Sjogren's syndrome. Arthritis Rheum 54, 1501-1508.
Elliott, P. J., Zollner, T. M., and Boehncke, W. H. (2003). Proteasome inhibition: a new anti-inflammatory strategy. J Mol Med (Berl) 81, 235-245.
Etienne, S., Gaborit, N., Henrionnet, C., Pinzano, A., Galois, L., Netter, P., Gillet, P., and Grossin, L. (2008). Local induction of heat shock protein 70 (Hsp70) by proteasome inhibition confers chondroprotection during surgically induced osteoarthritis in the rat knee. Biomed Mater Eng 18, 253-260.
Feng, B., Zhang, Y., Mu, J., Ye, Z., Zeng, W., Qi, W., Luo, Z., Guo, Y., Yang, X., and Yuan, F. (2010). Preventive effect of a proteasome inhibitor on the formation of accelerated atherosclerosis in rabbits with uremia. J Cardiovasc Pharmacol 55, 129-138.
Fineschi, S., Reith, W., Guerne, P. A., Dayer, J. M., and Chizzolini, C. (2006). Proteasome blockade exerts an antifibrotic activity by coordinately down-regulating type I collagen and tissue inhibitor of metalloproteinase-1 and up-regulating metalloproteinase-1 production in human dermal fibroblasts. FASEB J 20, 562-564.
Fissolo, N., Kraus, M., Reich, M., Ayturan, M., Overkleeft, H., Driessen, C., and Weissert, R. (2008). Dual inhibition of proteasomal and lysosomal proteolysis ameliorates autoimmune central nervous system inflammation. Eur J Immunol 38, 2401-2411.
Goldberg, A. L., and Rock, K. L. (1992). Proteolysis, proteasomes and antigen presentation. Nature 357, 375-379.
Gomez, A. M., Vrolix, K., Martinez-Martinez, P., Molenaar, P. C., Phernambucq, M., van der Esch, E., Duimel, H., Verheyen, F., Voll, RE., Manz, R. A., et al. (2011). Proteasome inhibition with bortezomib depletes plasma cells and autoantibodies in experimental autoimmune myasthenia gravis. J Immunol 186, 2503-2513.
Hosseini, H., Andre, P., Lefevre, N., Viala, L., Walzer, T., Peschanski, M., and Lotteau, V. (2001). Protection against experimental autoimmune encephalomyelitis by a proteasome modulator. J Neuroimmunol 118, 233-244.
Ichikawa, H. T., Conley, T., Muchamuel, T., Jiang, J., Lee, S., Owen, T., Barnard, J., Nevarez, S., Goldman, B. I., Kirk, C. J., et al. (2012). Beneficial effect of novel proteasome inhibitors in murine lupus via dual inhibition of type I interferon and autoantibody-secreting cells. Arthritis Rheum 64, 493-503.
Inoue, S., Nakase, H., Matsuura, M., Mikami, S., Ueno, S., Uza, N., and Chiba, T. (2009). The effect of proteasome inhibitor MG132 on experimental inflammatory bowel disease. Clin Exp Immunol 156, 172-182.
Jin, Y., Xu, L., Wu, X., Feng, J., Shu, M., Gu, H., Gao, G., Zhang, J., Dong, B., and Chen, X. (2019). Synergistic Efficacy of the Demethylation Agent Decitabine in Combination With the Protease Inhibitor Bortezomib for Treating Multiple Myeloma Through the Wnt/beta-Catenin Pathway. Oncol Res 27, 729-737.
Koca, S. S., Ozgen, M., Dagli, F., Tuzcu, M., Ozercan, I. H., Sahin, K., and Isik, A. (2012). Proteasome inhibition prevents development of experimental dermal fibrosis. Inflammation 35, 810-817.
Kortum, K. M., Mai, E. K., Hanafiah, N. H., Shi, C. X., Zhu, Y. X., Bruins, L., Barrio, S., Jedlowski, P., Merz, M., Xu, J., et al. (2016). Targeted sequencing of refractory myeloma reveals a high incidence of mutations in CRBN and Ras pathway genes. Blood 128, 1226-1233.
Kramer, U., Illig, T., Grune, T., Krutmann, J., and Esser, C. (2007). Strong associations of psoriasis with antigen processing LMP and transport genes TAP differ by gender and phenotype. Genes Immun 8, 513-517.
Lang, V. R., Mielenz, D., Neubert, K., Bohm, C., Schett, G., Jack, H. M., Voll, R E., and Meister, S. (2010). The early marginal zone B cell-initiated T-independent type 2 response resists the proteasome inhibitor bortezomib. J Immunol 185, 5637-5647.
Lee, S. J., Levitsky, K., Parlati, F., Bennett, M. K., Arastu-Kapur, S., Kellerman, L., Woo, T. F., Wong, A. F., Papadopoulos, K. P., Niesvizky, R., et al. (2016). Clinical activity of carfilzomib correlates with inhibition of multiple proteasome subunits: application of a novel pharmacodynamic assay. Br J Haematol 173, 884-895.
Ma, Y., Chen, B., Liu, D., Yang, Y., Xiong, Z., Zeng, J., and Dong, Y. (2011). MG132 treatment attenuates cardiac remodeling and dysfunction following aortic banding in rats via the NF-kappaB/TGFbeta1 pathway. Biochem Pharmacol 81, 1228-1236.
Manier, S., Salem, K. Z., Park, J., Landau, D. A., Getz, G., and Ghobrial, I. M. (2017). Genomic complexity of multiple myeloma and its clinical implications. Nat Rev Clin Oncol 14, 100-113.
Mutlu, G. M., Budinger, G. R, Wu, M., Lam, A. P., Zirk, A., Rivera, S., Urich, D., Chiarella, S. E., Go, L. H., Ghosh, A. K., et al. (2012). Proteasomal inhibition after injury prevents fibrosis by modulating TGF-beta(1) signalling. Thorax 67, 139-146.
Nair, P. M., Starkey, M. R., Haw, T. J., Liu, G., Horvat, J. C., Morris, J. C., Verrills, N. M., Clark, A. R, Ammit, A. J., and Hansbro, P. M. (2017). Targeting PP2A and proteasome activity ameliorates features of allergic airway disease in mice. Allergy 72, 1891-1903.
Neubert, K., Meister, S., Moser, K., Weisel, F., Maseda, D., Amann, K., Wiethe, C., Winkler, T. H., Kalden, J. R., Manz, R A., et al. (2008). The proteasome inhibitor bortezomib depletes plasma cells and protects mice with lupus-like disease from nephritis. Nat Med 14, 748-755.
Niewerth, D., Kaspers, G. J., Assaraf, Y. G., van Meerloo, J., Kirk, C. J., Anderl, J., Blank, J. L., van de Ven, P. M., Zweegman, S., Jansen, G., et al. (2014). Interferon-gamma-induced upregulation of immunoproteasome subunit assembly overcomes bortezomib resistance in human hematological cell lines. J Hematol Oncol 7, 7.
Orlowski, M. (1990). The multicatalytic proteinase complex, a major extralysosomal proteolytic system. Biochemistry 29, 10289-10297.
Park, J., Bae, E. K., Lee, C., Choi, J. H., Jung, W. J., Ahn, K. S., and Yoon, S. S. (2014). Establishment and characterization of bortezomib-resistant U266 cell line: constitutive activation of NF-kappaB-mediated cell signals and/or alterations of ubiquitylation-related genes reduce bortezomib-induced apoptosis. BMB Rep 47, 274-279.
Pawlyn, C., and Morgan, G. J. (2017). Evolutionary biology of high-risk multiple myeloma. Nat Rev Cancer 17, 543-556.
Podar, K., Gouill, S. L., Zhang, J., Opferman, J. T., Zorn, E., Tai, Y. T., Hideshima, T., Amiot, M., Chauhan, D., Harousseau, J. L., et al. (2008). A pivotal role for Mcl-1 in Bortezomib-induced apoptosis. Oncogene 27, 721-731.
Puttaparthi, K., and Elliott, J. L. (2005). Non-neuronal induction of immunoproteasome subunits in an ALS model: possible mediation by cytokines. Exp Neurol 196, 441-451.
Qiang, Y. W., Ye, S., Chen, Y., Buros, A. F., Edmonson, R., van Rhee, F., Barlogie, B., Epstein, J., Morgan, G. J., and Davies, F. E. (2016). MAF protein mediates innate resistance to proteasome inhibition therapy in multiple myeloma. Blood 128, 2919-2930.
Qiang, Y. W., Ye, S., Huang, Y., Chen, Y., Van Rhee, F., Epstein, J., Walker, B. A., Morgan, G. J., and Davies, F. E. (2018). MAFb protein confers intrinsic resistance to proteasome inhibitors in multiple myeloma. BMC Cancer 18, 724.
Rivett, A. J. (1989). The multicatalytic proteinase of mammalian cells. Arch Biochem Biophys 268, 1-8.
Sakairi, T., Hiromura, K., Takahashi, S., Hamatani, H., Takeuchi, S., Tomioka, M., Maeshima, A., Kuroiwa, T., and Nojima, Y. (2011). Effects of proteasome inhibitors on rat renal fibrosis in vitro and in vivo. Nephrology (Carlton) 16, 76-86.
Savvidou, I., Khong, T., Cuddihy, A., McLean, C., Horrigan, S., and Spencer, A. (2017). beta-Catenin Inhibitor BC2059 Is Efficacious as Monotherapy or in Combination with Proteasome Inhibitor Bortezomib in Multiple Myeloma. Mol Cancer Ther 16, 1765-1778.
Schmidt, N., Gonzalez, E., Visekruna, A., Kuhl, A. A., Loddenkemper, C., Mollenkopf, H., Kaufmann, S. H., Steinhoff, U., and Joeris, T. (2010). Targeting the proteasome: partial inhibition of the proteasome by bortezomib or deletion of the immunosubunit LMP7 attenuates experimental colitis. Gut 59, 896-906.
Singh, A. V., Bandi, M., Aujay, M. A., Kirk, C. J., Hark, D. E., Raje, N., Chauhan, D., and Anderson, K. C. (2011). PR-924, a selective inhibitor of the immunoproteasome subunit LMP-7, blocks multiple myeloma cell growth both in vitro and in vivo. Br J Haematol 152, 155-163.
Sonneveld, P., Avet-Loiseau, H., Lonial, S., Usmani, S., Siegel, D., Anderson, K. C., Chng, W. J., Moreau, P., Attal, M., Kyle, R A., et al. (2016). Treatment of multiple myeloma with high-risk cytogenetics: a consensus of the International Myeloma Working Group. Blood 127, 2955-2962.
Tron, A. E., Belmonte, M. A., Adam, A., Aquila, B. M., Boise, L. H., Chiarparin, E., Cidado, J., Embrey, K. J., Gangl, E., Gibbons, F. D., et al. (2018). Discovery of Mcl-1-specific inhibitor AZD5991 and preclinical activity in multiple myeloma and acute myeloid leukemia. Nat Commun 9, 5341.
Turner, J. G., Kashyap, T., Dawson, J. L., Gomez, J., Bauer, A. A., Grant, S., Dai, Y., Shain, K. H., Meads, M., Landesman, Y., et al. (2016). XPO1 inhibitor combination therapy with bortezomib or carfilzomib induces nuclear localization of IkappaBalpha and overcomes acquired proteasome inhibitor resistance in human multiple myeloma. Oncotarget 7, 78896-78909.
van der Heijden, J. W., Oerlemans, R, Lems, W. F., Scheper, R. J., Dijkmans, B. A., and Jansen, G. (2009). The proteasome inhibitor bortezomib inhibits the release of NFkappaB-inducible cytokines and induces apoptosis of activated T cells from rheumatoid arthritis patients. Clin Exp Rheumatol 27, 92-98.
Vanderlugt, C. L., Rahbe, S. M., Elliott, P. J., Dal Canto, M. C., and Miller, S. D. (2000). Treatment of established relapsing experimental autoimmune encephalomyelitis with the proteasome inhibitor PS-519. J Autoimmun 14, 205-211.
Waiser, J., Budde, K., Schutz, M., Liefeldt, L., Rudolph, B., Schonemann, C., Neumayer, H. H., and Lachmann, N. (2012). Comparison between bortezomib and rituximab in the treatment of antibody-mediated renal allograft rejection. Nephrol Dial Transplant 27, 1246-1251.
Yang, L., Chen, J., Han, X., Zhang, E., Huang, X., Guo, X., Chen, Q., Wu, W., Zheng, G., He, D., et al. (2018). Pirh2 mediates the sensitivity of myeloma cells to bortezomib via canonical NF-kappaB signaling pathway. Protein Cell 9, 770-784.
Zhang, X. D., Baladandayuthapani, V., Lin, H., Mulligan, G., Li, B., Esseltine, D. W., Qi, L., Xu, J., Hunziker, W., Barlogie, B., et al. (2016). Tight Junction Protein 1 Modulates Proteasome Capacity and Proteasome Inhibitor Sensitivity in Multiple Myeloma via EGFR/JAK1/STAT3 Signaling. Cancer Cell 29, 639-652.

1. A method of treating a subject in need thereof with a blood disorder, comprising:
administering an effective amount of a LMP7-selective inhibitor to the subject, wherein the subject has a t(4:14) or t(14;16) translocation.

2. The method of claim 1, wherein the LMP7-selective inhibitor is a compound according to formula (I):

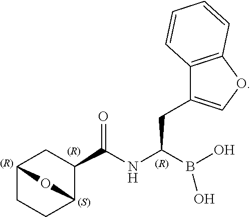

3. The method of claim 1, wherein the blood disorder is a premalignant condition.

4. The method of claim 3, wherein the blood disorder is monoclonal gammopathy of uncertain significance (MGUS): smoldering multiple myeloma (SMM); plasma cell leukemia and/or solitary plasmacytoma.

5. The method of claim 1, wherein the blood disorder is plasmacytoma and/or amyloid light-chain (AL) amyloidosis.

6. The method of claim 1, wherein the blood disorder is multiple myeloma.

7. The method of claim 1, wherein the subject shows an incomplete and/or suboptimal response to the administration of one or more pan-proteasome inhibitor.

8. The method of claim 1, wherein the subject is resistant to treatment with one or more pan-proteasome inhibitors.

9. The method of claim 1, wherein the subject is refractory to treatment with one or more pan-proteasome inhibitors.

10. The method of claim 7, wherein the one or more pan-proteasome inhibitors is selected from the group consisting of bortezomib, carfilzomib, and ixazomib.

11. The method of claim 1, wherein the subject has a blood disorder with a genetic alteration.

12. The method of claim 11, wherein the genetic alteration is a gene mutation, dysregulated gene expression, and/or gene dependency.

13. The method of claim 11, wherein the genetic alteration is in specific genes or pathways selected from the group consisting of IRF4, XPO1, MAX, MAF, MAFB, MCL1, FGFR3, IGF1R, CDKN2A, EGFR, Wnt/β-Catenin pathway, NFκB pathway, ubiquitination pathway, MAPK pathway, and/or DNA repair pathway.

14. The method of claim 11, wherein the genetic alteration is in one or more of the genes selected from the group consisting of APC, ARHGAP45, ASH2L, ATM, ATXN7, BRCA2, CCND2, CDC20, CDKN2A, CITED2, COQ6, DLST, DNAJC9, EGFR, EPC2, FGFR3, IGF1R, IRF2, IRF4, IRS1, KRAS, LYZ, MAF, MAP4K3, MAX, MCL1, MED8, MEF2C, MMSET, MTA2, NFKB1, NRAS, NSD2, PIM2, POU2AF1, PSMC1, RAD21, RICTOR, RORA, SEC13, THY1, TP53, UBA52, WNT1, WNT5B, XPO1, and ZBTB38.

15. The method of claim 1, further comprising administering an effective amount of one or more additional therapeutic agents to the subject in need thereof.

16. The method of claim 15, wherein the one or more additional therapeutic agents is an EGFR pathway inhibitor, MAPK pathway inhibitor, XPO1 inhibitor, a DNA repair pathway inhibitor, FGFR pathway inhibitor, PI3K/AKT/mTOR pathway inhibitor, and/or MCL1 inhibitor.

17. The method of claim 16, wherein the EGFR pathway inhibitor is selected from the group consisting of erlotinib, afatinib, gefitinib, cetuximab, panitumumab, lapatinib, osimertinib, trastuzumab, and/or pertuzumab.

18. The method of claim 16, wherein the MAPK pathway inhibitor is selected from the group consisting of trametinib, cobimetinib, binimetinib, selumetinib, refametinib, pimasertib, AMG 510, MRTX849, vemurafenib, dabrafenib, encorafenib, LXH254, HM95573, XL281, RAF265, RAF709, LY3009120, ulixertinib, SCH772984, TNO155, RMC-4630, JAB-3068, JAB-3312, AMG-510, MRTX849, LY3499446, and/or BI 1701963.

19. The method of claim 16, wherein the XPO1 inhibitor is selinexor and/or KPT-8602.

20. The method of claim 16, wherein the DNA repair pathway inhibitor is selected from the group consisting of M3541, M4076, BAY1895344, NOV1401, E7016, BGB-290, CEP-9722, Olaparib, Rucaparib, Niraparib, and/or Talazoparib.

21. The method of claim 16, wherein the FGFR pathway inhibitor is selected from the group consisting of erdafitinib, AZD4547, LY2874455, debio 1347, NVP-BGJ398, pemigatinib, rogaratinib, PRN1371, TAS-120, and/or nintedanib.

22. The method of claim 16, wherein the PI3K/AKT/mTOR pathway inhibitor is selected from the group consisting of rapamycin, temsirolimus, everolimus, ridaforolimus, alpelisib, idelalisib, copanlisib, duvelisib, MK-2206, and/or AZD5363.

23. The method of claim 16, wherein the MCL1 inhibitor is selected from the group consisting of A-1210477, VU661013, AZD5991, AMG-176, AMG-397, S63845, S64315, venetoclax, HDM201, NVP-CGM097, RG-7112, MK-8242, RG-7388, SAR405838, AMG-232, DS-3032, RG7775, and/or APG-115.

24. The method of claim 1, wherein the LMP7-selective inhibitor is administered orally.

25. The method of claim 1, wherein the LMP7-selective inhibitor is administered once a day.

26. A method of treating cancer in a subject in need thereof, comprising
administering an effective amount of an LMP7-selective inhibitor to the subject, wherein the subject has cancer with a genetic alteration.

27. The method of claim 26, wherein the LMP7 inhibitor is a compound according to formula (I):

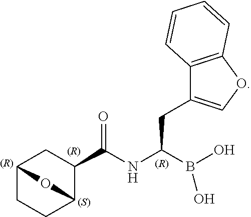

28. The method of claim 26, wherein the cancer is a solid tumor.

29. The method of claim 26, wherein the cancer is linked to chronic inflammation.

30. The method of claim 1, wherein the cancer is melanoma, glioma, glioblastomas, or cancer of the breast, lung, bladder, esophagus, stomach, colon, head, neck, ovary, prostate, pancreas, rectum, endometrium, or liver.

31. The method of claim 30, wherein the cancer is selected from the group consisting of triple-negative breast cancer, non-small cell lung cancer, and head and neck carcinoma.

32. The method of claim 26, wherein the cancer is a hematological malignancy.

33. The method of claim 32, wherein the hematological malignancy is selected from the group consisting of mantle cell lymphoma (MCL), T cell leukemia/lymphoma, acute myeloid leukemia (AML), acute lymphoblastic leukemia (ALL), diffuse large B-cell lymphoma (DLBCL), chronic lymphocytic leukemia (CLL), chronic myelogenous leukemia (CML), follicular lymphoma (FL), or marginal zone B-cell lymphoma (MZL).

34. The method of claim 32, wherein the hematological malignancy is selected from the group consisting of plasmacytoma, lymphoplasmacytic lymphoma, amyloid light-chain amyloidosis, and Waldenstrom's macroglobulinemia.

35. The method of claim 26, wherein the genetic alteration is a gene mutation, dysregulated gene expression, and/or gene dependency.

36. The method of claim 35, wherein the genetic alteration is in specific genes or pathways selected from the group consisting of IRF4, XPO1, MAX, MAF, MAFB, MCL1, FGFR3, IGF1R, CDKN2A, EGFR, Wnt/β-Catenin pathway, NFκB pathway, ubiquitination pathway MAPK pathway, and/or DNA repair pathway.

37. The method of claim 26, wherein the genetic alteration is in one or more of the genes selected from the group consisting of APC, ARHGAP45, ASH2L, ATM, ATXN7, BRCA2, CCND2, CDC20, CDKN2A, CITED2, COQ6, DLST, DNAJC9, EGFR, EPC2, FGFR3, IGF1R, IRF2, IRF4, IRS1, KRAS, LYZ, MAF, MAP4K3, MAX, MCL1, MED8, MEF2C, MMSET, MTA2, NFKB1, NRAS, NSD2, PIM2, POU2AF1, PSMC1, RAD21, RICTOR, RORA, SEC13, THY1, TP53, UBA52, WNT1, WNT5B, XPO1, and ZBTB38.

38. The method of claim 26, further comprising administering an effective amount of one or more additional therapeutic agents to the subject in need thereof.

39. The method of claim 38, wherein the one or more additional therapeutic agents is an EGFR pathway inhibitor, MAPK pathway inhibitor, XPO1 inhibitor, a DNA repair pathway inhibitor, FGFR pathway inhibitor, PI3K/AKT/mTOR pathway inhibitor, and/or MCL1 inhibitor.

40. The method of claim 39, wherein the EGFR pathway inhibitor is selected from the group consisting of erlotinib, afatinib, gefitinib, cetuximab, panitumumab, lapatinib, osimertinib, trastuzumab, and/or pertuzumab.

41. The method of claim 39, wherein the MAPK pathway inhibitor is selected from the group consisting of trametinib, cobimetinib, binimetinib, selumetinib, refametinib, pimasertib, AMG 510, MRTX849, vemurafenib, dabrafenib, encorafenib, LXH254, HM95573, XL281, RAF265, RAF709, LY3009120, ulixertinib, SCH772984, TNO155, RMC-4630, JAB-3068, JAB-3312, AMG-510, MRTX849, LY3499446, and/or BI 1701963.

42. The method of claim 39, wherein the XPO1 inhibitor is selinexor and/or KPT-8602.

43. The method of claim 39, wherein the DNA repair pathway inhibitor is selected from the group consisting of talazoparib, niraparib, olaparib, veliparib, rucaparib, pamiparib, AZD7648, M3814, CC-115, BAY1895344, AZD6738, M6620, M4344, M1774, M4076, M3541, AZD0157, AZD1390, prexasertib, GDC-0425, SRA-737, AZD1775, and/or Debio 0123.

44. The method of claim 39, wherein the FGFR pathway inhibitor is selected from the group consisting of erdafitinib, AZD4547, LY2874455, Debio 1347, NVP-BGJ398, pemigatinib, rogaratinib, PRN1371, TAS-120, and/or nintedanib.

45. The method of claim 39, wherein the PI3K/AKT/mTOR pathway inhibitor is selected from the group consisting of rapamycin, temsirolimus, everolimus, ridaforolimus, alpelisib, idelalisib, copanlisib, duvelisib, MK-2206, and/or AZD5363.

46. The method of claim 39, wherein the MCL1 inhibitor is selected from the group consisting of A-1210477, VU661013, AZD5991, AMG-176, AMG-397, S63845, S64315, venetoclax, HDM201, NVP-CGM097, RG-7112, MK-8242, RG-7388, SAR405838, AMG-232, DS-3032, RG7775, and/or APG-115.

47. The method of claim 26, wherein the LMP7-selective inhibitor is administered orally.

48. The method of claim 26, wherein the LMP7-selective inhibitor is administered once a day.
